(19) United States
(12) Patent Application Publication
(10) Pub. No.: US 2009/0287145 A1
(43) Pub. Date: Nov. 19, 2009

(54) DEVICES AND METHODS FOR TREATMENT OF ABDOMINAL AORTIC ANEURYSMS

(75) Inventors: Andrew H. Cragg, Edina, MN (US); Rodolfo C. Quijano, Laguna Hills, CA (US); Hosheng Tu, Newport Beach, CA (US); Stephen Sosnowski, Vista, CA (US); Robert J. Socci, JR., San Juan Capistrano, CA (US); George Wallace, Coto de Caza, CA (US)

Correspondence Address: KNOBBE MARTENS OLSON & BEAR LLP, 2040 MAIN STREET, FOURTEENTH FLOOR, IRVINE, CA, 92614, US

(73) Assignee: ALTURA INTERVENTIONAL, INC., San Clemente, CA (US)

(21) Appl. No.: 12/466,044

(22) Filed: May 14, 2009

Related U.S. Application Data
(60) Provisional application No. 61/053,378, filed on May 15, 2008.

(51) Int. Cl. A61M 29/00; A61M 31/00; A61F 2/04

(52) U.S. Cl. 604/96.01; 604/103.03; 623/23.7

(57) ABSTRACT

Methods and devices with two individual tubes for treating abdominal aortic aneurysm that bypass the aneurysm and are placed from the upper aorta to the iliac arteries. A separate upper cuff may also be provided, to secure the tubes above the aneurysm.

### CROSS-REFERENCE TO RELATED APPLICATIONS

This application is related to, and claims the benefit of U.S. Provisional 61/053,378 filed May 15, 2008, the entirety of which is hereby incorporated by reference herein and made a part of the present specification.

### BACKGROUND OF THE INVENTION

1. Field of the Invention

This invention relates generally to a modular biluminal endograft system for treatment of circumscribed dilatation of a large blood vessel, such as the abdominal aorta. More particularly, the present invention relates to the method of reducing the vessel diameter, minimizing possibility of vessel rupture and generating multiple lumina for down-stream flow continuity.

2. Description of the Related Art

The aorta delivers blood and oxygen to all arterial branches of the body, and as such is the largest artery of the human body. Based on the location of any particular segment in relation to the diaphragm, the aorta is referred to as thoracic or abdominal. The thoracic aorta if subdivided further into the ascending thoracic, that contains the aortic root and a tubular section containing the vessels leading to the brain, and the descending thoracic aorta. The abdominal aorta begins at the diaphragm and is terminal at the aortoiliac bifurcation where the arteries irrigating the lower limbs begin, and along its course giving off various visceral branches mesenteric arterial branches as well as the renal arteries. The diameter of the aorta varies along the different segments. The normal diameter of the thoracic aorta is in the order of about 3 cm at the tubular ascending portion, 2.5 cm at the descending thoracic aorta and 2 cm in the infrarenal abdominal aorta. The aortic dimensions vary relatively to body surface area, age and gender, males having larger aortic dimensions than females.

An enlargement of the aorta beyond its normal diameter is termed an aneurysm. The term aneurysm means dilation or dilatation. A segment of the aorta is termed aneurismal if its maximal diameter is greater than 1.5 times that of the adjacent proximal normal segment. Aortic aneurysms are more common in the abdominal aorta, one reason for this is that elastin, the principal load bearing protein present in the wall of the aorta, is reduced in the abdominal aorta as compared to the thoracic aorta (nearer the heart). Another is that the abdominal aorta does not possess vasa vasorum which hinders repair. Most are true aneurysms that involve all three layers (tunica intima, tunica media and tunica adventitia), and are generally asymptomatic before rupture.

The prevalence of abdominal aortic aneurysms (AAAs) increases with age, with an average age of 65-70 at the time of diagnosis. AAAs have been attributed to atherosclerosis, though other factors are involved in their formation. An AAA may remain asymptomatic indefinitely. There is a large risk of rupture once the size has reached 5 cm, though some AAAs may swell to over 15 cm in diameter before rupturing. Before rupture, an AAA may present as a large, pulsatile mass above the umbilicus. A bruit may be heard from the turbulent flow in a severe atherosclerotic aneurysm or if thrombosis occurs. Unfortunately, however, rupture is usually the first hint of AAA. Once an aneurysm has ruptured, it presents with a classic pain-hypotension-mass triad. The pain is classically reported in the abdomen, back or flank. It is usually acute, severe and constant, and may radiate through the abdomen to the back.

The diagnosis of an abdominal aortic aneurysm can be confirmed at the bedside by the use of ultrasound. Rupture could be indicated by the presence of free fluid in potential abdominal spaces, such as Morrison's pouch, the splenorenal space, subdiaphragmatic spaces and peri-vesical spaces. A contrast-enhanced abdominal CT scan is needed for confirmation. Only 10-25% of patients survive rupture due to large pre- and post-operative mortality. Annual mortality from ruptured abdominal aneurysms in the United States alone is about 15,000. Another important complication of AAA is formation of a thrombus in the aneurysm.

The definitive treatment for an aortic aneurysm is surgical repair of the aorta. This typically involves opening up of the dilated portion of the aorta and insertion of a synthetic (Dacron or Gore-tex) patch tube. Once the tube is sewn into the proximal and distal portions of the aorta, the aneurysmal sac is closed around the artificial tube. Instead of sewing, the tube ends, made rigid and expandable by Nitinol wireframe, can be much more simply and quickly inserted into the vascular stumps and there permanently fixed by external ligature.

In the recent years, the endoluminal treatment of abdominal aortic aneurysms has emerged as a minimally invasive alternative to open surgery repair. In endovascular surgery, a synthetic graft (stent-graft consisting of a polyester tube inside a metal cylinder) is attached to the end of a thin tube (catheter) that is inserted into the bloodstream, usually through an artery in the leg. Watching the progress of the catheter on an X-ray monitor, the surgeon threads the stent-graft to the weak part of the aorta where the aneurysm is located. Once in place, the graft is expanded. The stent-graft reinforces the weakened section of the aorta to prevent rupture of the aneurysm. The metal frame is expanded like a spring to hold tightly against the wall of the aorta, cutting off the blood supply to the aneurysm. The blood now flows through the stent-graft, avoiding the aneurysm. The aneurysm typically shrinks over time. This technique has been reported to have a lower mortality rate compared to open surgical repair, and is now being widely used in individuals with co-morbid conditions that make them high risk patients for open surgery. Some centers also report very promising results for the specific method in patients that do not constitute a high surgical risk group.

There have also been many reports concerning the endovascular treatment of ruptured abdominal aortic aneurysms, which are usually treated with an open surgery repair due to the patient's impaired overall condition. Mid-term results have been quite promising. The continuous development of the available stent technology in conjunction with the growing experience of the vascular experts that apply the technique will further enhance its safety and efficacy in the years to come. However, according to the latest studies, the current stent-grafts and procedures do not carry any overall survival benefit.

U.S. Pat. No. 5,676,697 issued on Oct. 14, 1997, entire contents of which are incorporated herein by reference, discloses an intraluminal graft for installing an intraluminal graft in relation to a bifurcation of a trunk vessel into two branch vessels to bypass an aneurysm defect or injury, wherein the intraluminal graft is formed of two cooperating graft prostheses.

The market today is populated by devices approximately 20F and greater requiring the need for a surgical cut-down approach utilizing catheters, guidewires and accessory devices which substantially eliminate the need for open surgical intervention. Although the cut-down approach significantly reduces the acute complications that often accompany open surgical intervention, the ultimate goal and the market trend is to reduce delivery system profiles and to be able to perform the procedure of delivering an endograft percutaneously, which eliminates the need for the cut-down procedure. There is a clinical need for addressing the endoleak and device anchoring/migration issues to benefit the AAA patient with new product design and features with a modular biluminal endograft system.

### SUMMARY OF THE INVENTION

The present invention overcomes the disadvantages associated with larger endograft as briefly described above.

In accordance with preferred embodiments of the present invention, some aspects of the invention relate to a modular biluminal endograft system for treatment of circumscribed dilatation of a large blood vessel, such as the abdominal aorta. One aspect of the present invention relates to the method of reducing the vessel diameter, minimizing possibility of vessel rupture and generating multiple lumina for down-stream flow continuity.

Some aspects of the invention provide a flexible or shapeable stent graft for inserting into a blood vessel, comprising a distal section, a proximal section and a graft body connecting the distal and proximal sections, the graft having an inner layer of water-tight flexible tube, a middle layer of semi-rigid or rigid material, and an outer layer of water-tight flexible overlap, wherein the graft is characterized with at least two water-tight layers. In one embodiment, the stent graft only has the middle layer and outer layer. In another embodiment, the middle layer comprises semi-rigid or rigid material in mesh-like or spiral configuration.

Some aspects of the invention provide a radially expandable sheath as a guiding sheath, comprising a continuous integral sheath body that is radially expandable under outward forces, wherein the radially expandable sheath is characterized with substantially little or no axial stretchability from a first configuration of a compressed state to a second configuration of an expanded state and vice versa.

Some aspects of the invention provide an endograft system for treatment of AAA, comprising a cuff and at least two endograft units, each endograft unit having a proximal end and a distal end, wherein the endograft units are made of compressible water-tight foam tubes having the proximal ends placed and fixed/secured at the cuff and the distal ends placed and fixed/secured in each of iliac arteries. In one embodiment, the first proximal end of a first endograft is at a substantial distance proximal to the second proximal end of a second endograft.

Some aspects of the invention provide an endograft for treatment of AAA comprising an impermeable section for excluding blood communication between a lumen of the endograft and a surrounding aneurysmal sac, and a porous section configured for placement across a renal artery ostium.

Some aspects of the invention provide an endograft for treatment of AAA comprising a neck attachment section, a graft body, and two leg sections, the neck attachment section having a multiple-anchoring mechanism that comprises at least a first anchoring element for placement at proximal to a renal artery and a second anchoring element axially spaced apart from the first anchoring element for placement at distal to the renal artery.

Some aspects of the invention provide an endograft for treatment of AAA comprising a neck attachment section, a first foam tube having a length to extend from the neck attachment section to a first iliac artery for fixation inside the first iliac artery, and a second form tube having a length to extend from the neck attachment section to a second iliac artery for fixation inside the second iliac artery, wherein both foam tubes are secured to the neck attachment section.

Some aspects of the invention provide a balloon endograft comprising: a neck attachment member, a body and two bifurcated distal ends, wherein the endograft comprises double layers and a space between the layers, the space being configured to be filled with fluid or hardenable foam to inflate the balloon endograft.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1A shows detailed structure of a D-graft.

FIG. 1B shows a pair of D-grafts with opposite charged magnets embedded in the facing surfaces of the two D-grafts.

FIG. 1C shows two grafts that are self-sealing even when placed asymmetrically.

FIG. 1D shows a pair of D-grafts with anchoring barbs.

FIGS. 2A and 2B show embodiments of an endograft in a compressed state configured for catheter or sheath delivering.

FIGS. 3A-3C show a radially expandable sheath for delivering an endograft.

FIGS. 4A-4C show schematics of placing a hemostatic cuff on an expandable sheath to advance an endograft into a blood vessel.

FIGS. 5A-5C show steps of advancing an endograft through an iliac artery to the aorta.

FIGS. 6A-6C illustrate one method for placing a neck subassembly of an endograft over a renal stent in-situ.

FIGS. 7A-7D illustrate one method for placing a neck subassembly of an endograft in-situ.

FIGS. 8A-8C illustrate one method of bypassing the renal arteries when implanting an AAA endograft.

FIGS. 9A-9D illustrate one method for placing an endograft and a renal stent for treatment of AAA.

FIGS. 10A-10E illustrate an alternate method for placing an endograft and a renal stent for treatment of AAA.

FIG. 11 shows an embodiment of an endograft for treatment of AAA.

FIG. 12 shows one embodiment of a stent graft with a double-neck attachment element for treating abdominal aortic aneurysms.

FIG. 13 shows one embodiment of a stent graft with coated surface for treating abdominal aortic aneurysms.

FIGS. 14A-14F show procedural steps for positioning a system for treating abdominal aortic aneurysms.

FIG. 15 shows a detailed proximal section of the stent graft system in FIG. 14E.

FIG. 16A shows a “double D” sponge plug to provide interlocked seal in blood vessel.

FIG. 16B shows a “ribbed sponge” plug to provide interlocked seal in blood vessel.

FIGS. 17A-17C show a sponge plug that is: (A) reinforced or supported with anchor structures; (B) with a radiopaque marker; and (C) with a radiopaque body.

FIG. 18 shows various configurations of a sponge plug.

FIG. 19 shows a delivery system for inserting soft, thrombogenic ‘pipe-cleaner’ like soft filler material into AAA sac.

FIG. 20 shows a delivery system for pulling the ‘pipe-cleaner’ like soft filler material into AAA sac by a tip mechanism.

FIGS. 21A-21C show a delivery system for pulling the ‘pipe-cleaner’ like soft filler material into AAA sac by a repositionable snare that may be located in a second lumen of a dual-lumen delivery catheter.

FIGS. 22A-22B show a delivery system for inserting the ‘pipe-cleaner’ like soft filler material into AAA sac by a balloon in a double lumen delivery catheter.

FIG. 23 shows a delivery system for squeezing the ‘pipe-cleaner’ like soft filler material into AAA sac by a nozzle delivery catheter.

FIGS. 24A-24B show comparison of: (A) a conventional AAA device and (B) an improved AAA device of the present invention.

FIGS. 25A-25C show an embodiment of an endograft made of curable foam tubes.

FIGS. 26A and 26B show a side-view and a top-view of a tubular graft comprising cuffs at each end, wherein the cuff has prongs that hold the graft in place when deployed.

FIGS. 27A-27D show a device for creation of a low-profile, percutaneous delivery, endoleak resistant vascular graft having inflatable ends and/or an inflation body.

FIGS. 28A-28F show a double-walled, baffled tube filled with a hardening or form-filling material with sufficient hoop strength to obviate the use of another support structure such as a metallic stent.

FIG. 29 shows a cuff construct with multiple through lumens so that multiple channels can be formed.

FIGS. 30A-30D show a method for introducing cuffs and endografts for treatment of AAA in an aortic area.

FIG. 31 shows one embodiment of an endograft made of double layer inflatable balloon without metal or rigid supporting component.

FIG. 32 shows one embodiment of an endograft made of two double layer inflatable balloon bodies without metal or rigid/stiff supporting component.

### DETAILED DESCRIPTION OF THE PREFERRED EMBODIMENT

The preferred embodiments of the present invention described below relate particularly to a device system or as a component/subassembly in a system for use in treating or repairing aneurysms. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

The aorta is the largest artery in a body, and it carries blood away from a heart. The aorta runs through the chest, where it is called the thoracic aorta. When it reaches an abdomen, it is called the abdominal aorta. The abdominal aorta supplies blood to the lower part of the body. Just below the abdomen, the aorta splits into two branches that carry blood into each leg. When a weak area of the abdominal aorta expands or bulges, it is called an abdominal aortic aneurysm (AAA). The pressure from blood flowing through your abdominal aorta can cause a weakened part of the aorta to bulge, much like a balloon. A normal aorta is about 1 inch (or about 2.5 centimeters) in diameter. However, an AAA can stretch the aorta beyond its safety margin. Aneurysms are a health risk because they can burst or rupture. AAA can cause another serious health problem. Clots or debris can form inside the aneurysm and travel to blood vessels leading to other organs in your body. If one of these blood vessels becomes blocked, it can cause severe pain or even more serious problems, such as limb loss. Abdominal aortic aneurysms are most often found when a physician is performing an imaging test, such as an abdominal ultrasound, computed tomography (CT) scan, or magnetic resonance imaging (MRI).

Systems for treating or repairing aneurysms such as abdominal aortic aneurysms and thoracic aortic aneurysms come in many forms. A typical system includes an anchoring and/or sealing component which is positioned in healthy tissue above the aneurysm and one or more grafts which are in fluid communication with the anchoring and/or sealing component and extend through the aneurysm and anchor in healthy tissue below the aneurysm. Essentially, the grafts are the components of the system that are utilized to establish a fluid flow path from one section of an artery to another section of the same or different artery, thereby bypassing the diseased portion of the artery. Essentially, the endovascular graft of the present invention comprises a number of components that make up a modular system. Although the overall endovascular graft comprises a number of components, the challenges associated with these types of systems include profile, flexibility and accessibility. The primary failure modes for a percutaneous device for treating abdominal aortic aneurysms include failure to access, rupture, endoleak with AAA expansion, migration or displacement of the device, AAA expansion, endoleak, and the like. The device integrity issues clinically include, among others, suture break, endoleaks, migration, iliac limb separation, stent graft fractures, proximal kink, and separation of cranial position of the graft.

### Mate-able Pair of Grafts

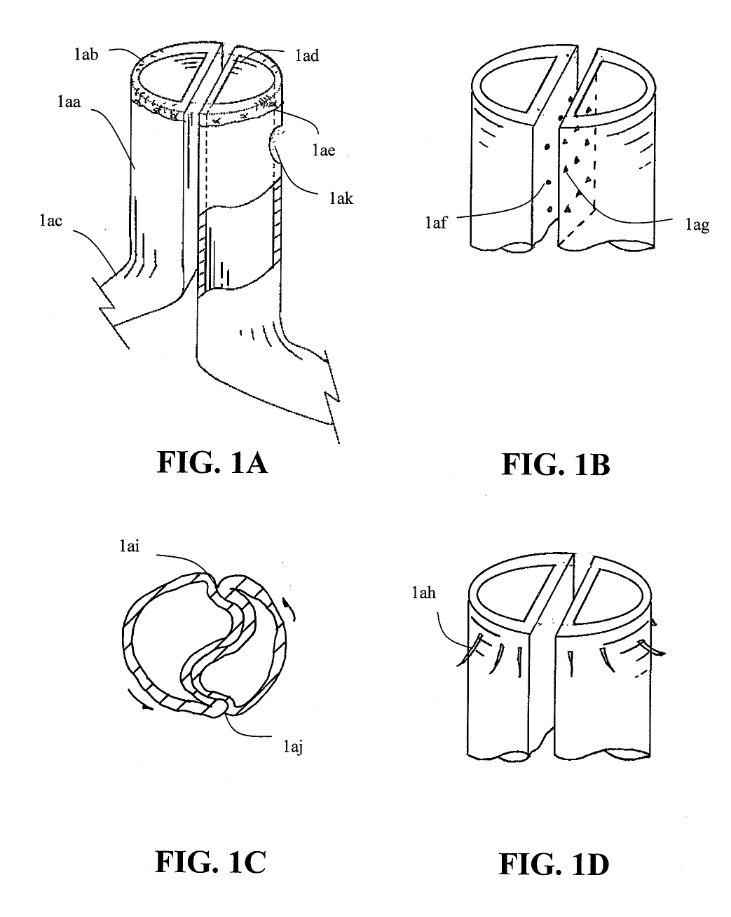

A stent graft for treating EVAR (endovascular aneurysm repair) problems of an abdominal aortic aneurysm may include features such as, low introductory profile, short neck, long leg/short leg catheterization, graft sizing, graft construction and the like. In one preferred embodiment, elements of a stent graft may comprise at least two layers, including a middle layer of a flat sheet, spiral, or mesh of laser cut elastic or semi-rigid material (for example, metal, Nitinol metal, shape memory metal, plastic, shape memory plastic or other flexible material), and an outer layer of expanded PTFE overwrap. Optionally, the stent graft further comprises a third inner layer of a stretchable expanded PTFE (polytetrafluoroethylene) tube. The layers are compacted to serve as the building material for the stent graft composite. The distal section (1ac) of the stent graft can be shaped to fit the graft into iliac artery. The stent graft can be shaped in different configurations, such as a D-shaped graft (D-graft) having a semi-circular like side and a flat side (FIG. 1A). In one embodiment, the expanded PTFE is impermeable to liquid or water. The inner PTFE layer and the outer PTFE layer serves to assure liquid-tightness of the composite constructing material.

Two D-shaped stent grafts of the present invention can form a cylindrical-like tubular appearance when two flat sides of the grafts face each other or mate intimately against each other. In one embodiment, the sleeve at the end (1ab) of the stent graft (1aa) can be formed by inverting the inner PTFE tube (1ad). In a further embodiment, the inverted portion of the PTFE tube can be secured to the middle layer or the inner portion of the inner layer by any fastening means, such as suturing (1ae), stapling, gluing, bonding, and the like. In one embodiment, the inner layer and the outer layer may use polyester fabric material (for example, Dacron) or other suitable material, such as substantially water-tight microfibers in woven form. In a further embodiment, the D-graft comprises an opening (1ak) for blood flow into a renal artery, wherein the opening may be created prior to implantation or be created by a wire piercing after the D-graft is placed in-situ, followed optionally by balloon expansion. It is important that the opening receives and matches the outer circumference of the renal stent graft intimately and water-proof to prevent endoleak.

In operations, each D-shaped graft may be loaded in the sheath of a delivery apparatus so that the first D-shaped graft can be accurately deployed in a mated fashion against the second D-shaped graft. In one preferred embodiment, the grafts are inserted into aorta via bilateral femoral sheaths. The grafts may be rotated to match the flat sides against each other and mate. In one embodiment, the flat sides of the two D-grafts are manually maneuvered or rotated so they face each other. In another embodiment, the mate-able sides (as illustrated in FIG. 1A) are manually maneuvered so they face each other. In one embodiment, at least a portion of the flat side of the grafts is embedded with rare-earth magnets with positive charge (1af) on one graft surface and negative charge (1ag) on the opposite graft surface to ensure control seal (for example, liquid-tight seal) and intimate contact of that portion when mating (FIG. 1B). In another embodiment, there is provided means for creating positive charged magnet at a first surface of the first graft and negative charged magnet at a second conformable surface of the second graft for intimate mating purposes. The conformable surface may be flat as in a D-graft.

In another embodiment, barbs can be incorporated and spaced apart appropriately at about the proximal portion of the D-shaped graft so that the barbs (1ah) would be deployed radially outwardly to anchor the graft at the aorta (FIG. 1D). In one embodiment, the barbs are generally sized and configured to allow the graft to move in an advancing direction with little resistance, whereas the barbs would engage into the aorta when the graft starts to move in a reversed direction. In another embodiment, the barbs are configured with a spring property so that the barbs extend outwardly (for example, spring-out) when the graft is deployed from the sheath. In still another embodiment, the barbs are made of shape memory material or temperature-sensitive material so that the barbs are activated at a threshold elevated temperature via hot saline or other electrical, chemical or biological means. In still another embodiment, the grafts are self-sealing or self-mating even when placed asymmetrically (FIG. 1C), wherein a portion of the contact surfaces mates against each other. The grafts as shown in FIG. 1C may comprise a pair of form tube grafts or other radially expandable grafts that result in intimate seal at the region between the two points (1ai and 1aj). The intimate seal region may be at about the proximal ends of the grafts or at proximity distal to the proximal ends. The grafts may be oversized so to intimately contact the arterial wall to seal the grafts and prevent blood leakage (endoleak).

D-grafts allow a non-custom method of supra vena EVAR by separating treatment of both renal arteries. Position of renal ostia in D-graft can be changed to accommodate varying anatomy. Complete EVAR can be performed with only two components selected for diameter (proximal and distal), length and renal ostia, when desired. For example, one can select a first D-graft having a length of 160 mm, a distal diameter of 26 mm, a proximal diameter of 16 mm, and a renal ostia about 20 mm proximal to the distal end and a second D-graft having a length of 140 mm, a distal diameter of 26 mm, a proximal diameter of 12 mm, and a renal ostia 10 mm proximal to the distal end. In the above examples, the proximal end of the second D-graft may lie at a plane distal to the proximal end of the first D-graft.

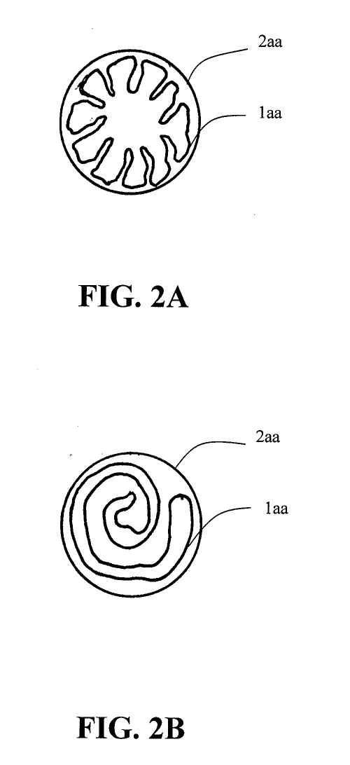

Sheet technology allows D-graft (1aa) to be better compressed for introduction into a smaller sheath (2aa) by rolling a graft as shown in FIGS. 2A and 2B. The cross-section of a D-graft may be transitional along its length from D-configuration in aorta section and to circular or circular-like configuration in iliac section. This transitional configuration can be accomplished by changing from a partial elastic member to a circumferential member in the graft construction longitudinally. The D-graft in aorta section can be configured with flexible multi-segments to accommodate delivering through or positioning at a tortuous blood vessel.

Some aspects of the invention relate to a flexible stent graft for inserting into a blood vessel, comprising a distal section, a proximal section and a graft body with a lumen that connects the distal and proximal sections, the graft having a first layer of flexible rigid or semi-rigid material, and a second layer of water-tight flexible overlap, wherein the graft is collapsible and is characterized with a low profile during the inserting operation. In one embodiment, the first layer comprises a spiral wire that is compressible within a sheath during the inserting operation. In another embodiment, the second layer invaginates onto the first layer after the first layer is positioned in place. In still another embodiment, the stent graft further comprises a third layer of water-tight flexible tube, wherein the graft is characterized with at least two water-tight layers, wherein the third layer is made of stretchable PTFE tube and the second layer is made of stretchable PTFE overlap.

One aspect of the invention relates to a flexible stent graft, wherein a sleeve at an end of the stent graft is formed by inverting an extra length of the third layer over the first and second layers. In one embodiment, the inverted sheath is secured to the first layer by fastening means for securing the inverted sheath with the first layer, the fastening means comprising suturing, stapling, gluing, or bonding. In another embodiment, the third layer is made of flexible fabrics or polymer tube and the second layer is made of flexible fabrics or polymer overlap. In still another embodiment, the second layer or the third layer is made of substantially water-tight microfibers woven material.

One aspect of the invention relates to a flexible stent graft, wherein barbs are incorporated and spaced apart appropriately at about the proximal section of the stent graft configured for anchoring the graft at wall of a blood vessel, wherein the barbs may be made of shape memory material or temperature-sensitive material. In one embodiment, anchors are provided at about the proximal section of the graft configured for anchoring the graft at wall of a blood vessel as a secondary operation.

Some aspects of the invention relate to a stent graft system comprising a first and a second stent grafts, the graft having an inner layer of stretchable expanded PTFE tube, a middle layer of semi-rigid or rigid material, and an outer layer of stretchable expanded PTFE overlap, wherein the proximal section of either stent graft is shaped to have a semi-circular like side and a mating side, wherein the first mating side of the first stent graft mates and matches intimately the second mating side of the second stent graft when the proximal sections of the two grafts are mated against each other to form a cylindrical-like tubular configuration. In one embodiment, the first distal section of the first stent graft is flexible for inserting into a right iliac artery and the second distal section of the second stent graft is flexible for inserting into a left iliac artery. In another embodiment, the first mating side of the first stent graft is configured to have positive charged magnet and the opposite second mating side of the second stent graft is configured to have negative charged magnet so to ensure control seal and intimate contact upon been mated. In still another embodiment, the proximal sections of the two stent grafts in the cylindrical-like tubular configuration are radially expandable to intimately fit and secure to the blood vessel.

In one embodiment, the first mating side is configured to have positive charged magnet and the opposite second mating side is configured to have negative charged so to ensure control seal and/or intimate contact.

In one embodiment, a sleeve at an end of the stent graft is formed by inverting the inner PTFE tube, wherein, the inverted PTFE tube is secured to the middle layer by fastening means for securing purposes, such as suturing, stapling, gluing, and bonding.

In one embodiment, the PTFE layers of the present invention are replaced by layers made of other flexible fabrics or polymers, for example, polyester fabrics or substantially water-tight microfibers.

In one embodiment, barbs are incorporated and spaced apart appropriately at about the proximal portion of the stent graft so that the barbs would be deployed radially outwardly to anchor the graft at the aorta wall. In a further embodiment, the barbs are made of shape memory material or temperature-sensitive material so that the barbs are activated or deployed at a threshold elevated temperature.

### Sheath Subassembly

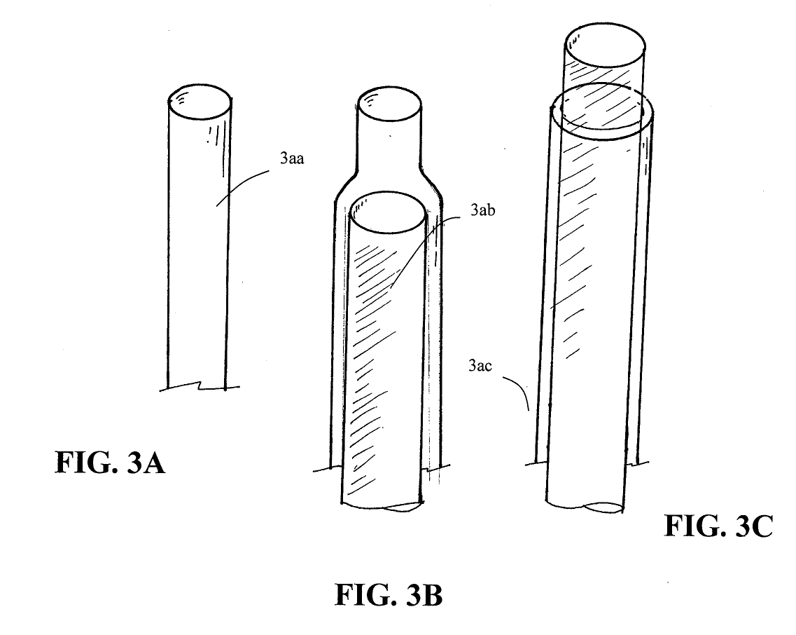

One aspect of the invention relates to an expandable flexible sheath. In one embodiment, the flexible sheath is configured radially expandable when needed. FIGS. 3A-3C show a radially expandable sheath, with substantially little or no axial stretchability/compressibility, comprising a continuous integral sheath that can be radially expanded under outward force. The expandable flexible sheath can be made of elastomeric polymer with embedded non-stretchable fibers or threads that are oriented substantially axially to exert limitation on axial stretchability. In one embodiment, the flexible sheath at its contracted state (3aa) can pass through tortuous or small diameter vessels, followed by inserting a larger device (3ab) through the sheath. Thus, this expandable sheath allows placement of a larger device like an endograft or D-graft of the invention through tortuous or smaller diameter vessels where advancement of a large sheath may be impossible, impassable, unpractical or may cause dissection. After placement of the large device, the expanded sheath (3ac) can be removed or retracted out of the patient. In one embodiment, the expandable flexible sheath is radially retractable. An expandable flexible sheath may function as a “guiding sheath”.

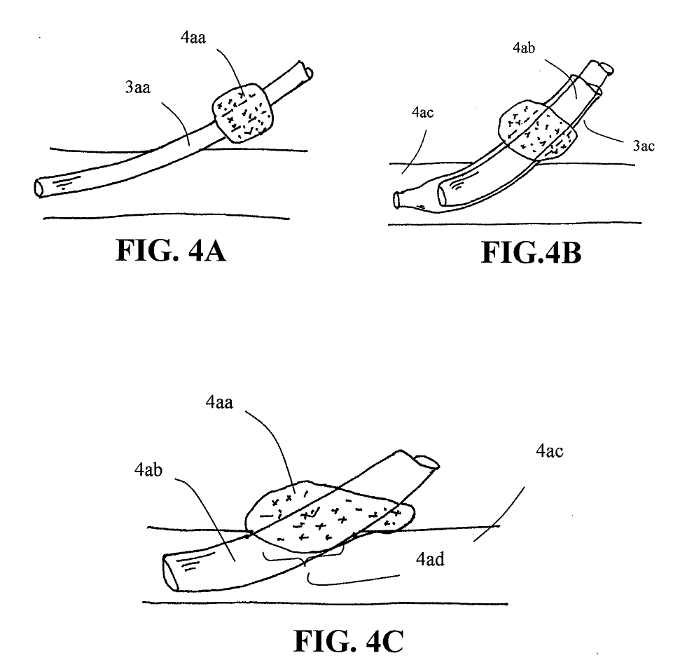

FIGS. 4A-4C show schematics of placing a hemostatic cuff (4aa) on the expandable sheath (3aa) at its retracted state that is configured for advancing the endograft (4ab) into a blood vessel (4ac). After the endograft is in place, the expanded sheath (3ac) is removed while the hemostatic cuff is positioned over the endograft at about the opening (4ad) of the blood vessel (4ac) temporarily.

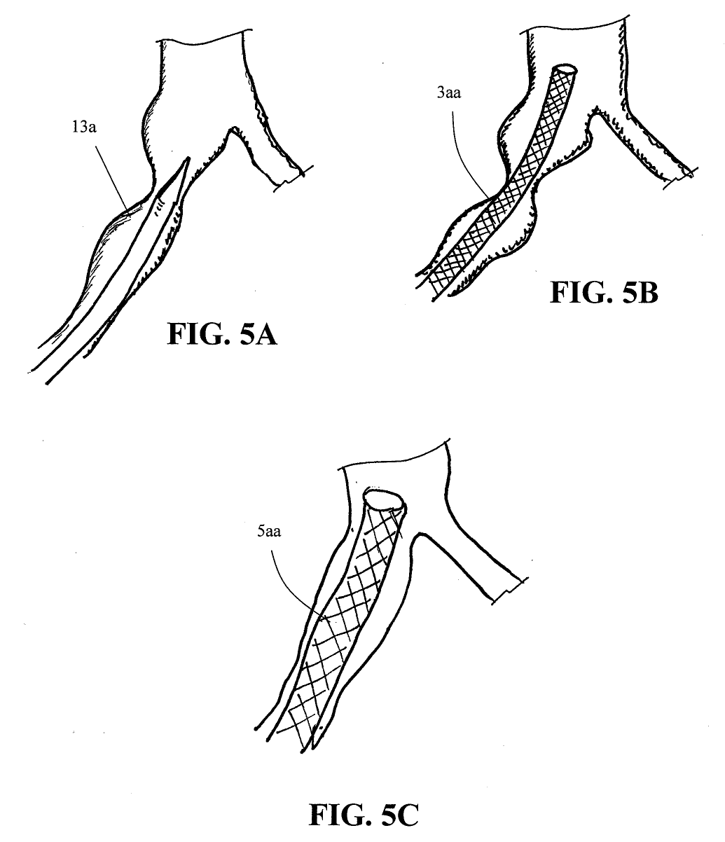

FIG. 5 shows steps of advancing an endograft through an iliac artery (13a) to the aorta. With small or stenotic iliac arteries, it may be impossible or unsafe to advance a large sheath. Therefore an expandable sheath at its small state (3aa) is advanced and then radially expanded to allow passage of large devices (5aa) as illustrated in FIG. 5A to FIG. 5C.

Some aspects of the invention provide a radially expandable sheath as a guiding sheath, comprising a continuous integral sheath body with a thin wall that is radially expandable under outward forces, wherein the radially expandable sheath is characterized with substantially little or no axial stretchability or contraction from a first configuration of a compressed state to a second configuration of an expanded state and vice versa.

A method of temporarily placing a hemostatic cuff at an incision of a blood vessel when inserting an endograft into a patient, the method comprising: (a) loading the hemostatic cuff on the expandable sheath of claim 1 at the first configuration; (b) inserting the compressed sheath through the incision into the blood vessel; (c) advancing the endograft into the blood vessel via a sheath lumen to expand the sheath to the second configuration; (d) holding the hemostatic cuff at proximity of the incision; and (e) removing the expanded sheath after the endograft and the cuff are properly positioned in place.

### The Neck Subassembly

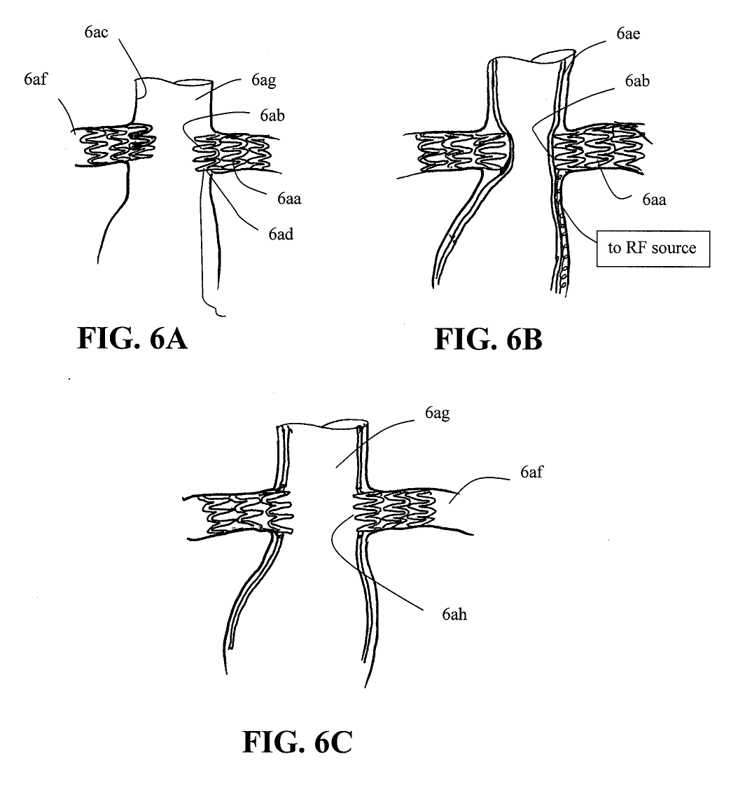

In one embodiment for a short neck endograft application, renal stent grafts could be implanted in the renal arteries, wherein the metal mesh portion of the renal stent graft is removably connected to an RF electrode (6ad) that is electrically connected to an outside RF source. As shown in FIG. 6A, the exposed end (6ab) of the renal stent graft (6aa) extends or protrudes beyond the aorta inner wall (6ac). FIG. 6B shows an endograft (6ae) being positioned inside the aorta, whereas the exposed end of the renal stent graft (6aa) contacts and presses against the external surface of the endograft intimately. By applying RF current to the stent edge (6ab), a hole (6ah) is created in endograft fabric (shown in FIG. 6C) for blood communication between the aorta (6ag) and the renal arteries (6af). The endograft is intimately and tightly pressed against the boundary of the renal artery ostium to prevent blood leakage or seepage.

Some aspects of the invention provide a method for placing an endograft for treatment of AAA while preserving blood communication from aorta to renal arteries, comprising: (a) placing a renal stent inside a renal artery, wherein a first end of the renal stent is inside the renal artery whereas the second end protrudes beyond the renal artery ostium; (b) placing the endograft in the AAA area, wherein the endograft intimately contacts the renal artery; (c) applying RF energy to the second end of the renal stent so to create a hole by RF energy and to protrude the renal stent into a lumen of the endograft. In one embodiment, the endograft comprises a pair of D-grafts. In another embodiment, the endograft comprises a pair of grafts with mate-able proximal sections.

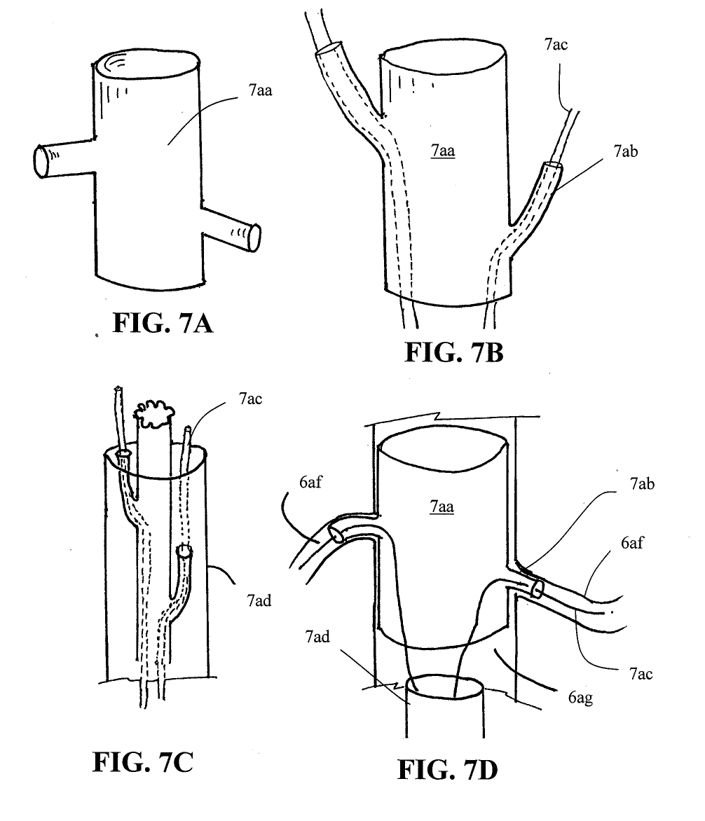

FIG. 7 illustrates one method for placing a neck subassembly of an endograft. As shown in FIG. 7A, one may use common polymer physical data to create an elastomeric graft cast construct (7aa) of juxta-renal aorta. The material used may be porous, biocompatible, durable and elastomeric. The construction could be similar to a rapid prototype process. In a second step shown in FIG. 7B, limbs (7ab) are compressed and cast with gelatin. Guide tubes are inserted to accept wires (7ac) for the construct (7aa). In operations, the construct (7aa) is compressed and loaded in a delivery sheath (7ad) as shown in FIG. 7C. The construct is thereafter released about the renal artery region, whereas each limb (7ab) is inserted into the renal artery (6af) via the guide wires introduction (see FIG. 7D).

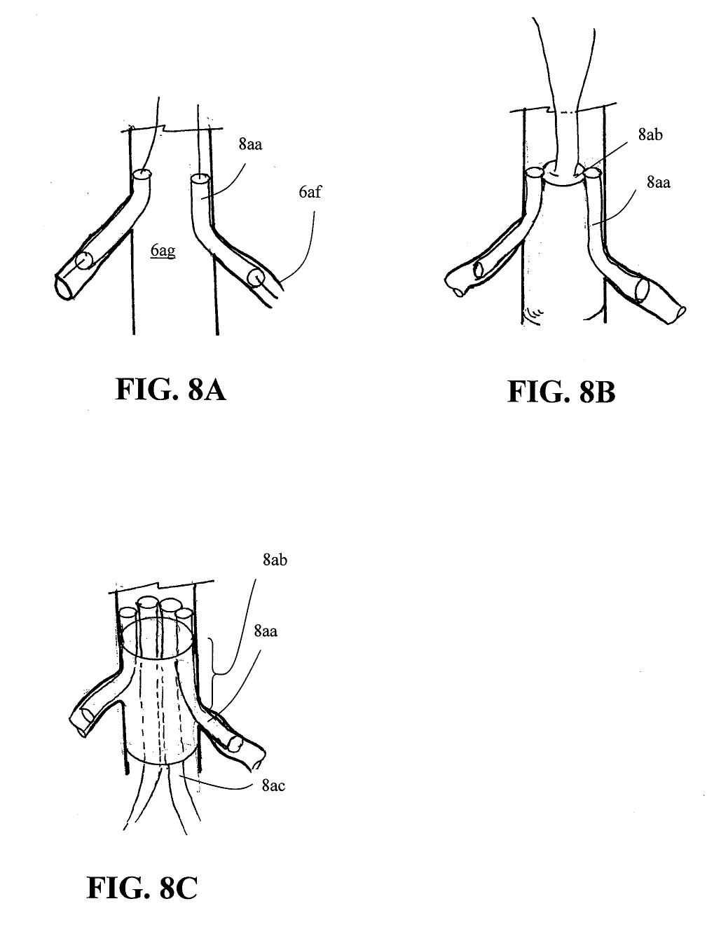

FIG. 8 illustrates one method of bypassing the renal arteries when implanting an AAA endograft. As shown in FIG. 8A, a tubular stent graft (8aa) from brachial artery is implanted about the aorta and renal region, wherein the distal end is inserted into the renal artery (6af) and the proximal end stays inside the aorta (6ag). Juxta-renal foam cuff (8ab) is then applied to the proximal ends of the implanted stent grafts (8aa) under supra-mesenteric fixation (shown in FIG. 8B). A pair of aorto-iliac grafts (8ac) as the endograft is then inserted through the cuff (shown in FIG. 8C), whereas the distal end of the aorto-iliac graft is inserted into the iliac artery. The justa-renal foam cuff is sized and configured to avoid migration, endoleak or blockage to normal blood flow.

One aspect of the invention provides an endograft system for treatment of AAA, comprising a cuff and four endograft units, each endograft unit having a proximal end and a distal end, all four proximal ends are placed and fixed at the cuff whereas a first distal end extends and is fixed in right renal artery, a second distal end extends and is fixed in left renal artery, a third distal end extends and is fixed in right iliac artery and a fourth distal end extends and is fixed in left iliac artery. In one embodiment, the endograft system isolates blood from flowing into or in fluid communication with the aneurysmal zone as means for preventing endoleak.

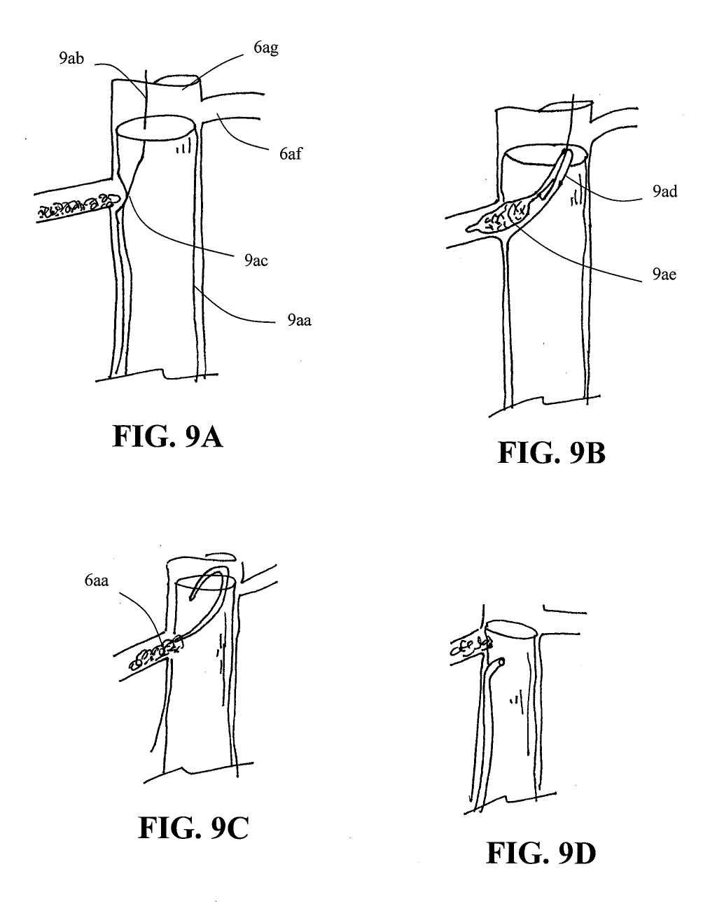

FIG. 9 illustrates one method for placing an endograft and a renal stent for treatment of AAA in a patient. In operations, an endograft (9aa) is placed in an aorta (6ag) over renal arteries (6af). FIG. 9A shows that a wire (9ab) is inserted to pierce the graft at about the renal artery region (9ac). FIG. 9B shows that a special dual lumen stent catheter (9ad) is used to push through the graft at the piercing point (9ac). Thereafter, the balloon (9ae) of the dual lumen stent catheter is inflated to create lumen for the renal stenting operation, wherein one end of the renal stent (6aa) is placed inside the renal artery and the other end is within the aorta (as shown in FIGS. 9C and 9D).

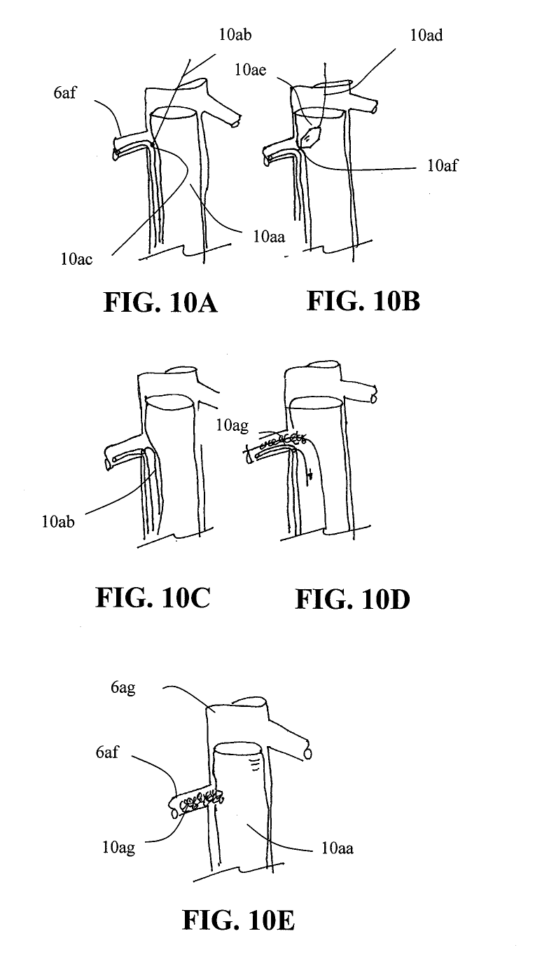

FIG. 10 illustrates an alternate method for placing an endograft and a renal stent for treatment of AAA. In operations, a graft (10aa) is placed in an aorta (6ag) over renal arteries (6af). FIG. 10A shows that a wire (10ab), preferably with a sharp end, is inserted to pierce the graft at about the renal artery region (10ac). FIG. 10B shows that a special 2-lumen guide catheter (10ad) is used, wherein the second lumen accepts wire to pierce through the graft at the piercing point (10ac). Thereafter, the balloon (10ae) of the 2-lumen guide catheter is inflated to create orifice (10af) for the renal artery. The curved wire is inserted in the guide and is pulled down to center the orifice (as shown in FIG. 10C). Subsequently, the renal artery is catheterized and stented (as shown in FIG. 10D), wherein one end of the renal stent (10ag) is placed inside the renal artery and the other end is within the endograft (as shown in FIG. 10E).

Some aspects of the invention provide a method for placing an endograft for treatment of AAA while preserving blood communication from aorta to renal arteries, comprising: (a) placing a renal stent inside a renal artery, wherein a first end of the renal stent is inside the renal artery whereas the second end is positioned at about the renal artery ostium; (b) placing the endograft in the AAA area, wherein the endograft intimately and compressively contacts the renal artery ostium; (c) providing a wire at about the ostium site and piercing through the endograft so to create a hole into the renal artery configured for blood communication from aorta to the renal artery. In one embodiment, the method is followed by another step of balloon expansion at about the hole to enlarge the hole size.

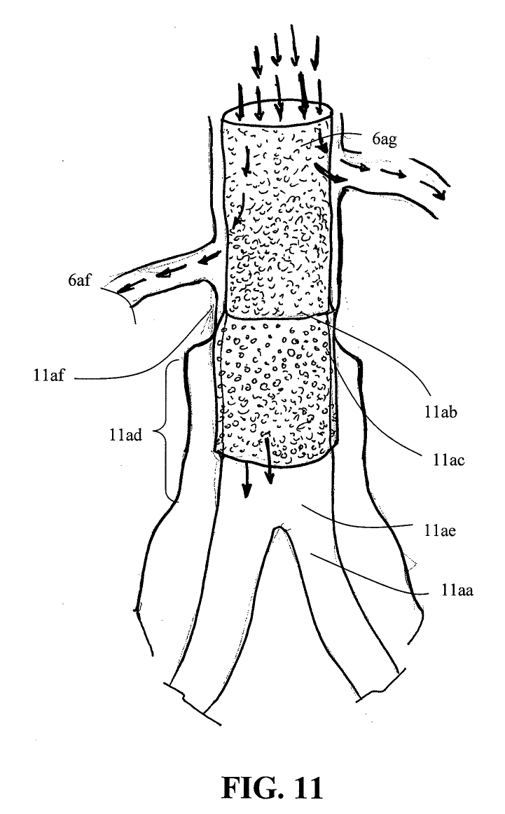

FIG. 11 shows an alternate endograft for treatment of AAA. The endograft (11aa) comprises an impermeable section that begins from a proximal end (11ac) of the endograft located below the renal artery ostia (11af) and extends into the iliac arteries, and a porous section placed across renal arteries. The porous section may be created by securing a macro-porous sleeve (11ab) over the impermeable section, for example, an overlap zone (11ad) extending from the proximal end (11ac) to the distal end (11ae) of the porous sleeve. Thus blood could flow from aorta (6ag) to renal arteries via the porous sleeve and to iliac arteries via the endograft while bypassing the aneurismal zone.

Some aspects of the invention relate to an endograft for treatment of an abdominal aortic aneurysm (AAA) comprising an impermeable section for excluding blood communication between a lumen of the endograft and a surrounding aneurysmal sac, and a porous section configured for placement across a renal artery ostium. In one embodiment, the endograft comprises a macro-porous sleeve that is longer than the impermeable section, the porous section being created by securing the macro-porous sleeve over at least a portion of the impermeable section.

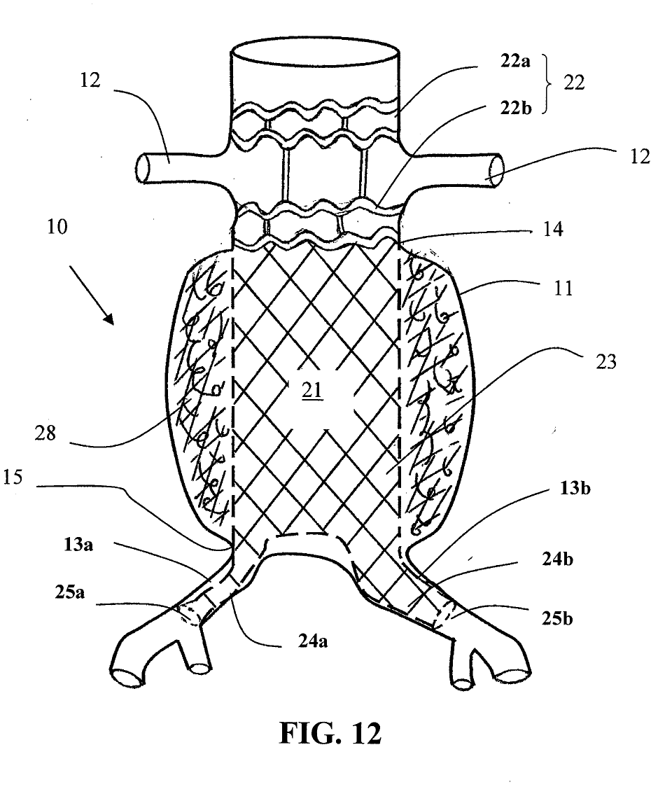

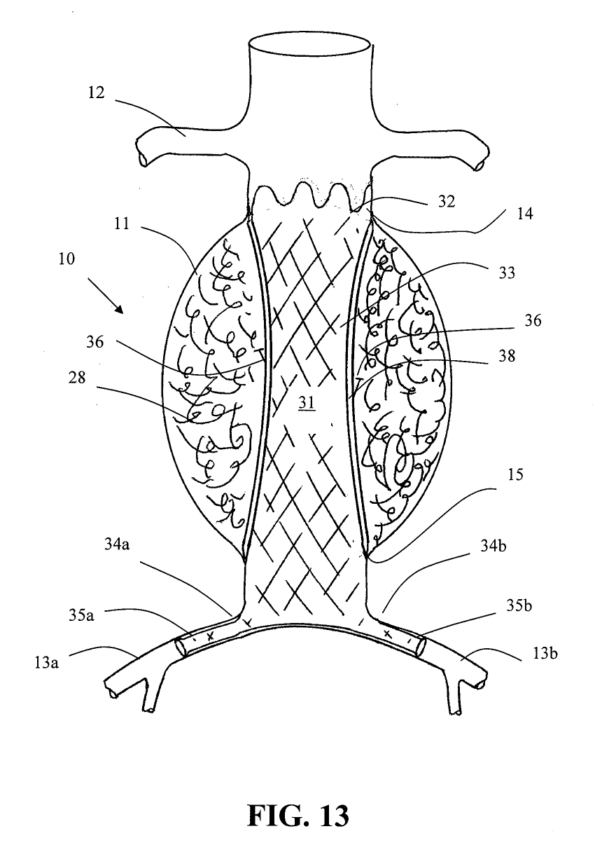

FIGS. 12-14 show one or another alternate embodiment of a stent graft or endograft, system, and methods of use for treatment of abdominal aortic aneurysms. Specifically, FIG. 12 shows one embodiment of stent grafts (21) of the present invention to be percutaneously deployed into the aneurismal aorta region (10) for implantation. In one embodiment, the stent graft (21) comprises a neck attachment section (22), the graft body or trunk (23), and two leg sections (24a), (24b). The neck attachment section (22) may comprise a single neck attachment element (32) as shown in FIG. 13 or a double neck attachment element (22a) and (22b) shown in FIG. 12. In the exemplary embodiment after delivering the graft to the position, the neck attachment element is radially expandable that is sized and configured to contact intimately the tissue of the aortic wall for securing the neck attachment section in place with little or no device migration. The securing operation may be accomplished by a number of barbs protruding therefrom for anchoring. The barbs can be configured to deploy outwardly in sync with the expansion of the neck attachment element. The single neck attachment element (32) may be mesh-like or porous (for example, without cloth covering or graft material) and is generally attached to the aorta distal to the renal arteries (12). The first (22a) of the double neck attachment element may be secured to the aorta proximal to at least one renal artery (12) while the second (22b) of the double neck attachment element is secured to the aorta distal to the renal artery. In one embodiment, the expanded diameter of the first of the double neck attachment is different from that of the second one.

For the neck attachment section with a single neck attachment element (32) as shown in FIG. 13 or a plural neck attachment element as shown in FIG. 12, the length of graft trunk distal to the attachment element for seal and fixation in aortic neck is appropriately sized and configured according to the determined diameter of aortic neck. Similarly, the length and diameter of each leg section for seal in the iliac artery is also appropriately sized and configured according to the determined native diameter of the iliac artery. In one preferred embodiment, the single neck attachment element (32) and/or the second neck attachment element (22b) of the double neck attachment elements may have the graft material integrally extending from the graft trunk.

U.S. Pat. No. 6,383,193 issued on May 7, 2002, entire contents of which are incorporated herein by reference, discloses a delivery system for the percutaneous insertion of a self-expanding vena cava filter device system, the system comprising constraining the filter in a compact condition within an elongated, radially flexible and axially stiff tubular member. The neck attachment section could be a shape memory wireframe that is axially rigid and radially expandable so that it can be much more simply and quickly inserted, deployed and there permanently fixed by associated external ligature, such as barbs or anchors on the wireframe. The wireframe may comprise a substantially zigzag pattern, mesh-like or other appropriate pattern suitable for radial expansion and anchoring.

A wireframe made from shape memory alloy may be deformed from an original, heat-stable configuration to a second, heat-unstable configuration. The application of a desired temperature causes the alloy to revert to an original heat-stable configuration. A particularly preferred shape memory alloy for this application is binary nickel titanium alloy (NiTi alloy) comprising about 55.8 percent Ni by weight, commercially available under the trade designation Nitinol. This NiTi alloy may be configured to undergo a phase transformation at physiological temperatures. A stent or wireframe made of this material is deformable when chilled. Thus, at low temperatures, for example, below twenty degrees centigrade, the stent is compressed so that it can be delivered to the desired location. The stent may be kept at low temperatures by circulating chilled saline solutions. The stent expands when the chilled saline is removed and when it is exposed to higher temperatures within the patient's body, generally around thirty-seven degrees centigrade.

The graft trunk (23), configured to anchor and seal the stent graft within a vessel and comprising a substantially tubular stent structure, can be an expandable tubular metal stent with graft material inside. The graft material or component may be made from any number of suitable biocompatible materials, including woven, knitted, sutured, extruded, or cast materials comprising polyester, polytetrafluoroethylene, silicones, urethanes, and ultra lightweight polyethylene, such as that commercially available under the trade designation Spectra™. The materials may be porous or nonporous. Exemplary materials include a woven polyester fabric made from Dacron™ or other suitable PET-type polymers which is folded to reduce its size and which is attached to one or both ends of a radially expandable stent by means of sutures or gluing. When the stent self-expands or is balloon expanded, the graft unfolds around the stent. In one embodiment, there is provided a porous endoluminal graft which is made of a spun matrix of polyurethane combined with a self-expanding stent. The elastomeric polyurethane fibers allow the graft to compress with the stent and thereby permit delivery of the stent-graft through a relatively small catheter.

Graft material is affixed to at least a portion of the trunk section (23) and all of the legs (24a, 24b). The graft material may be attached to various portions of the underlying structure by sutures. In one embodiment, the graft material is affixed with a continuous stitch pattern on the end of the trunk section (23) and by single stitches elsewhere. It is important to note that any pattern may be utilized and other devices, such as staples, may be utilized to connect the graft material to the underlying structure. The sutures may comprise any suitable biocompatible material that is preferably highly durable and wear resistant. In one embodiment, the graft trunk intimately contact the aorta at an upper contact region (14) and the lower contact region (15) to prevent blood from seeping into the aneurysmal region (11) of the abdominal aorta.

In the exemplary embodiment, the first (24a) of the leg section of the stent graft (21) is placed within the right common iliac artery (13a), wherein the distal end member (25a) of the first leg section (24a) is with a self-expandable or balloon expandable Nitinol wireframe. Similarly, the second leg section (24b) is inserted into the left common iliac artery (13b) with a self-expandable or balloon expandable distal end member (25b). After the stent graft is positioned and deployed in place, the aneurysmal region (28) of the aorta (outside of the core channel) may be further treated with foam embolization. The ends of the leg section (24a) and (24b) may be flared for better anchoring and sealing in the downstream arteries. The flared section may be formed by flaring the last portion of the stent element. The leg sections are the bypass conduits through which the blood flows in the aneurysmal section of the artery. By eliminating the blood flow to the diseased section, the pressure is reduced and thus there is less of a chance of the aneurysm rupturing.

Referring now to FIG. 13, there is illustrated an exemplary embodiment of an endograft or stent graft (31) with a graft trunk (33) having anchoring and sealing components at each end section in accordance with the present invention. In one embodiment, the stent graft (31) is characterized with a central trunk section that is gradually narrowed from either end section of the trunk (33). In another embodiment, the middle section of the graft trunk (33) is equipped with at least one foam-injecting port (36). The foam-injecting port can be a self-sealing site for accessing to a foam-containing catheter with a needle or a one-way valve for accessing to a foam-containing catheter with a blunt tip. In still another embodiment, the stent graft (31) is characterized with a polymer coat or polymer membrane (38) at an exterior surface of the trunk, wherein the polymer coat or membrane can be either thrombogenic to promote foam embolization or non-thrombogenic to mitigate foam adhesion to the stent graft.

Some aspects of the invention relate to an endograft for treatment of an abdominal aortic aneurysm (AAA) comprising a neck attachment section, a graft body, and a leg section, the neck attachment section having a multiple-anchoring mechanism that comprises at least a first anchoring element for placement at proximal to a renal artery and a second anchoring element axially spaced apart from the first anchoring element, wherein the second anchoring element is configured for placement at distal to the renal artery. In one embodiment, the multiple-anchoring mechanism comprises a third anchoring element configured for placement at about a region between two renal arteries.

One aspect of the invention relates to an endograft for treatment of AAA comprising a neck attachment section, a first foam tube having a proximal end and a length to extend from the neck attachment section to a first iliac artery for fixation inside the first iliac artery, and a second form tube having a proximal end and a length to extend from the neck attachment section to a second iliac artery for fixation inside the second iliac artery, wherein both foam tubes are secured to the neck attachment section. In one embodiment, the first proximal end of a first foam tube is located at a substantial distance proximal to the second proximal end of a second foam tube. In another embodiment, the neck attachment element comprises a hanger, and wherein the proximal end of the first foam tube is configured with a hook to securely couple the hook to the hanger. In still another embodiment, the proximal end of the first foam tube is magnetically coupled to the neck attachment element. In a preferred embodiment, a distal end of the first foam tube is flared to anchor and seal the distal end to surrounding tissue of the first iliac artery or wherein a distal end of the first foam tube is balloon expanded to anchor and seal the distal end to surrounding tissue of the first iliac artery, or wherein a distal end of the first foam tube is made of shape memory material to anchor and seal the distal end to surrounding tissue of the first iliac artery.

One aspect of the invention relates to an endograft, wherein a proximal section of the foam tubes is made of inflatable elements, and wherein the proximal section is distendable to anchor and secure the proximal section against wall of a blood vessel. In one embodiment, at least one of the foam tubes further comprises an inflatable tube body. In another embodiment, at least one of the foam tubes comprises a double-walled, baffled tube body filled with form-filling material that functions as a flexible graft with sufficient hoop strength to obviate use of a radial positioning structure. In still another embodiment, a portion of the baffled layer of at least one end of the foam tube is everted to create a cuff. In a preferred embodiment, an aneurysm sac of the AAA is filled with foam material that is subsequently hardened in situ, wherein the foam material is introduced via a one-way valve mounted on the first form tube into the aneurysm sac, and wherein the foam material is selected from the group consisting of polyvinyl alcohol foam, poly(ethylene-co-vinyl alcohol), cellulose acetate, poly(2-hydroxyethyl methacrylate), acrylates, and combinations thereof. The foam material is treated with UV light or heat in situ.

### The Cuff Subassembly

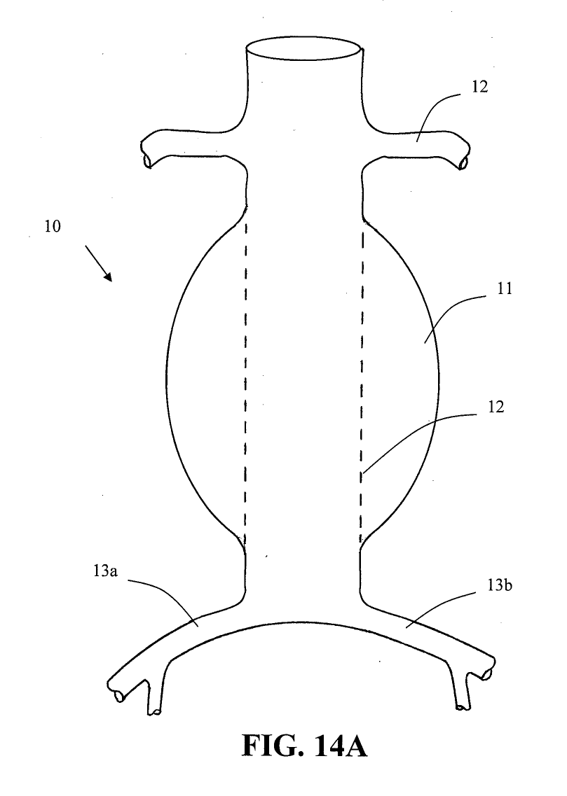

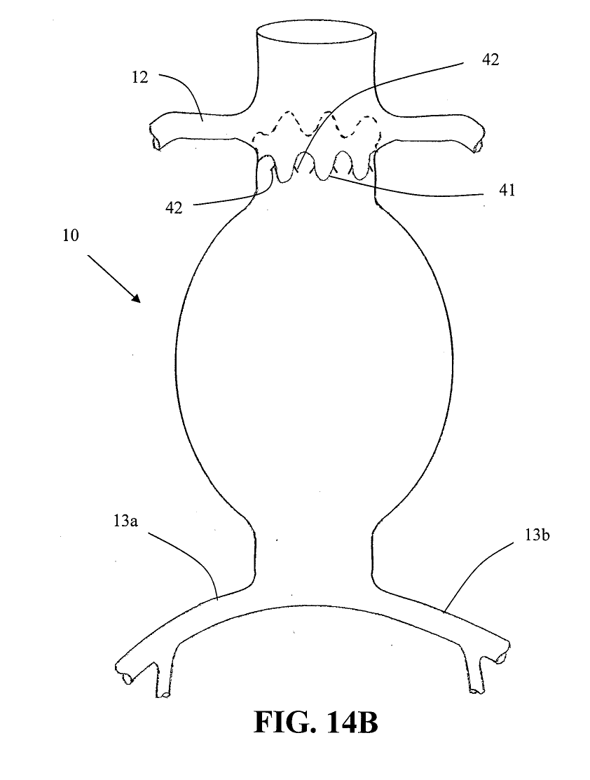

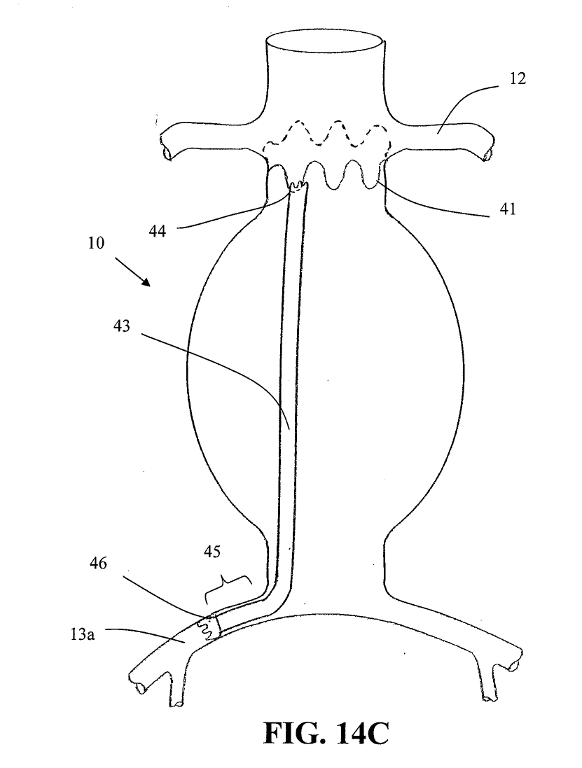

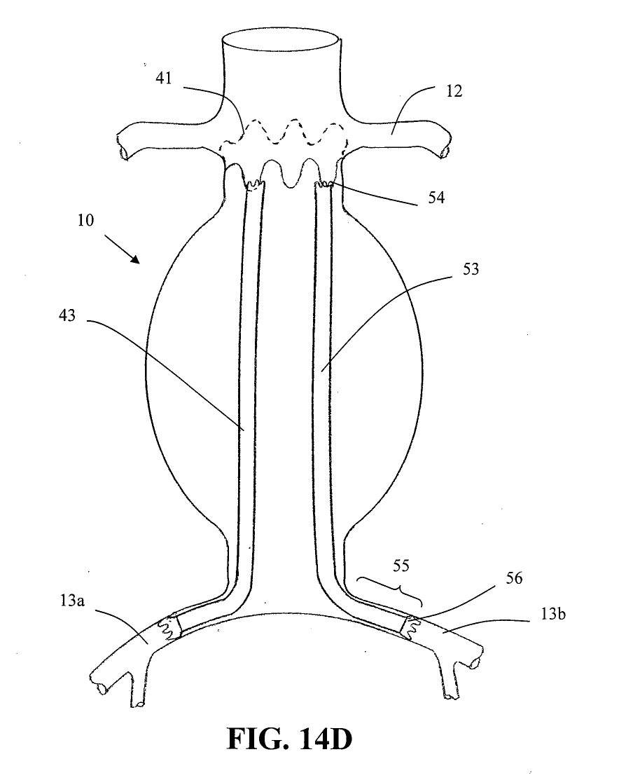

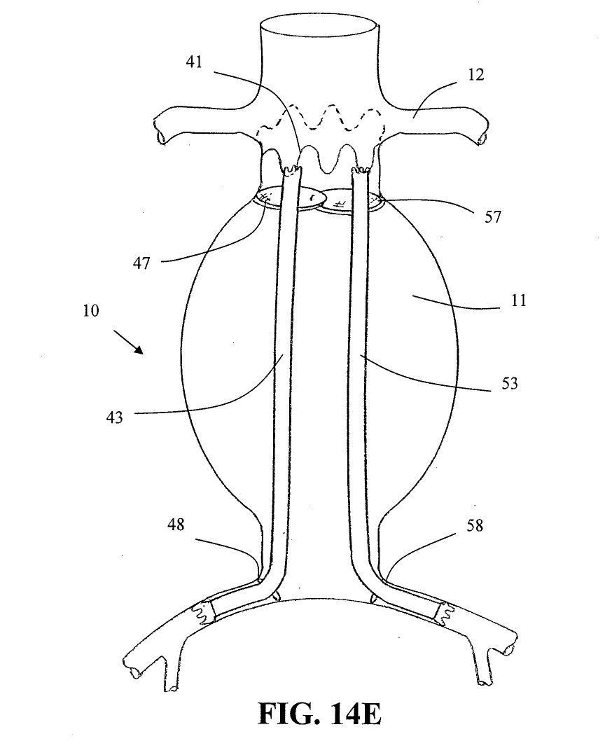

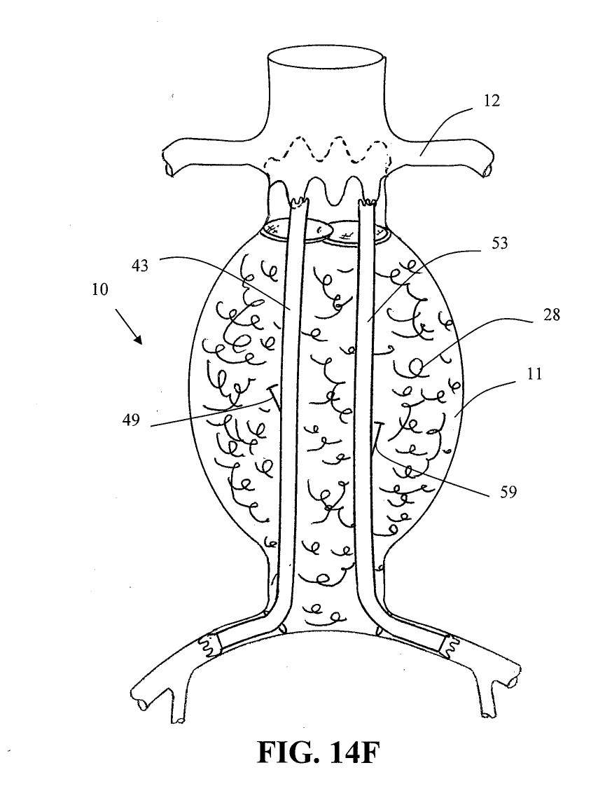

Referring now to FIG. 14, there is illustrated an exemplary embodiment of a modular biluminal endograft system with components and procedures for placing such a system in a body in accordance with the present invention. Some aspects of the invention relate to a method of repairing an abdominal aortic aneurysm in the arterial wall at about the aorta and the right and left iliac arteries comprising the steps of: (a) percutaneously introducing and advancing a guidewire into one of the right and left femoral arteries, into the respective one of the right and left iliac arteries and then into the lumen of the aorta beyond the area of the aneurysm; (b) assembling a neck attachment element in a collapsed state about a distal end segment of a first deployment catheter, wherein the first deployment catheter having a guidewire lumen formed therein adapted to be fitted over the guidewire; (c) delivering the neck attachment element to a site of the aorta close to the renal artery ostium to provide an attachment seat of predetermined size approximating the diameter of the aorta lumen beyond the area of the aneurysm; (d) deploying by means of self-expanding or balloon-expanding the neck attachment element to anchor or secure the element in place with, say barbs; (e) withdrawing the first deployment catheter; (f) assembling a first elongated tubular graft prosthesis about a distal end segment of a second deployment catheter, the first graft prosthesis having a continuous side wall extending between the distal end to the proximal end, wherein the graft prosthesis may be reinforced with metal mesh or stenting element at either end or both ends; (g) delivering the first graft prosthesis so the proximal end is positioned about the neck attachment element and the distal end about one of the right iliac artery; (h) deploying by means of anchoring the proximal end of the first graft prosthesis onto the neck attachment element while deploying the metal mesh in the right iliac artery; (i) percutaneously withdrawing the second deployment catheter; (j) repeating steps f to step i with a second elongated tubular prosthesis and the third deployment catheter and having a deployed metal mesh in the left iliac artery. In a preferred embodiment, the circumferential area next to the luminal openings of the proximal end of the two graft prostheses are sealed to prevent any blood from flowing into the exterior of the two prostheses in the abdominal aneurysm. In another preferred embodiment, the distal end is sized and configured, after deployment, to seal the graft lumen and iliac arteries from the aneurysm section.

As shown in FIG. 14A, the goal for treating an abdominal aortic aneurysm is to limit the blood flow in the abdominal aorta substantially constant by maintaining the blood flowing along about the dashed line (12). A second goal is to supply adequate blood volume to the iliac arteries (13a, 13b) from the thoracic artery by bypassing the aneurismal artery portion (11). In the exemplary embodiment as shown in FIG. 14B, the first step of procedures for positioning an endograft system is to percutaneously delivery a neck attachment element (41) to the healthy tissue above the aneurysm, but distal to the renal arteries (12). Thereafter, the neck attachment element is deployed in place with anchoring members, such as barbs (42).

In one embodiment, balloon expansion of the neck attachment element occurs at a pressure sufficient to cause the stent-like element to radially expand and to anchor the element to the surrounding tissue.

The second step is to percutaneously deliver a first tube (43) with adequate strength, flexibility and length as shown in FIG. 14C so the proximal end (44) of the tube (43) is secured to part of the neck attachment element (41) while the distal end section (45) is placed within the right iliac artery (13a). In one embodiment, the neck attachment element is equipped with a hanger (62) and the proximal end (44) of the first tube is configured with a hook (61) to securely couple the hook to the hanger. Other mechanisms of coupling, such as magnetic coupling or button-slot coupling may also be feasible. The distal end (46) of the first tube may be flared as discussed above, balloon expanded, or made of shape memory material to anchor and seal the distal end to the surrounding tissue.

Referring now to FIG. 14D, a second tube (53) with adequate strength, flexibility and length is percutaneously delivered to the abdominal aorta area so the proximal end (54) of the tube (53) is secured to part of the neck attachment element (41) while the distal end section (55) is placed within the left iliac artery (13b). As discussed above, the distal end (56) of the second tube may be flared, balloon expanded, or made of shape memory material to anchor and seal the distal end to the surrounding tissue.

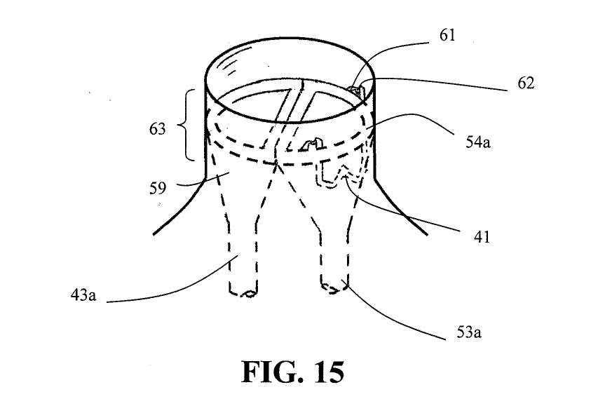

Before foam embolization is initiated, the aneurysmal aorta region (11) may be sealed from the rest of the blood flowing vessel. In one embodiment as shown in FIG. 14E, a first proximal sealing member (47) is provided to the first tube (43) and a second proximal sealing element (57) is provided to the second tube (53). The sealing elements (47, 57) are sized, configured and placed overlap to each other so to cover the open area beyond the tubes at about the upper healthy aorta region. The sealing members (47, 57) can be provided as an integral part of the tubes. In one preferred embodiment as shown in FIG. 15, the proximal ends (44a, 54a) of the tubes (43a, 53a) are configured to a trumpet shape (59) and sized to intimately occupy the space at about the neck fixation region (63) as shown in FIG. 15. In one embodiment, the trumpet shaped proximal end is expandable by using shape memory material.

In an alternate embodiment, the distal section is sealed against the vessel wall with a stopper (48, 58) for the first and second tubes (43, 53), respectively. Foam material can be introduced into the aneurysm (11) and hardened in situ (FIG. 14F). In this case, the foam material would stay in the aneurysm even without the proximal sealing members (47 and 57). In the exemplary embodiment, the foam material before hardened may be delivered through the tubes (43, 53) into the delivering ports (49 and 59). As discussed above, the delivering port can be a self-sealing site or have a one-way valve that is accessible to foam-containing catheters.

Some aspects of the invention relate to an endograft system with a neck anchoring mechanism and two foam tubes, wherein the blood bypasses the aneurysm via flowing through the foam tubes from upper aorta to iliac arteries. In one embodiment, the aneurysm is filled with foam material that is subsequently hardened in situ. In another embodiment, the foam material is introduced via a one-way valve mounted on the form tube into the aneurysm and is hardened thereafter in situ. The foam material may be polyvinyl alcohol foam, EVAL poly(ethylene-co-vinyl alcohol)), cellulose acetate, p-HEMA (poly(2-hydroxyethyl methacrylate)), acrylates, combinations thereof, and the like.

Polyvinyl alcohol foam (PAF) offers a number of advantages over other embolic material, including biocompatibility, promotion of progressive thrombosis and fibrosis, permanence, compressibility, and manageability. The clinical cases illustrate the kinds of lesions that are amenable to embolization, including arteriovenous malformations, arteriovenous fistulas, meningiomas, nasopharyngeal tumors, and particularly for AAA treatment.

A vascular implant formed of a compressible foam material has a compressed configuration from which it is expansible into a configuration substantially conforming to the shape and size of a vascular site to be embodied. Preferably, the implant is formed of a hydrophobic, macro porous foam material, having an initial configuration of a scaled-down model of the vascular site, from which it is compressible into the compressed configuration. The implant could be made by scanning the vascular site to create a digitized scan data set; using the scan data set to create a three-dimensional digitized virtual model of the vascular site; using the virtual model to create a scaled-down physical mold of the vascular site; and using the mold to create a vascular implant in the form of a scaled-down model of the vascular site. To embolism a vascular site, the implant is compressed and passed through a delivery catheter, the distal end of which has been passed into a vascular site. Upon entering the vascular site, the implant expands in situ substantially to fill the vascular site. A retention element is contained within the catheter and has a distal end detachably connected to the implant. A flexible, tubular deployment element is used to pass the implant and the retention element through the catheter, and then to separate the implant from the retention element when the implant has been passed out of the catheter and into the vascular site. In one preferred embodiment, the compressible foam material is injected as a transportable moving material that is solidified in-situ and substantially conforms to the shape and size of a vascular site to be embodied.

### Endo-Plug

PVA sponge with different porosities (for example, 700, 300, 30 microns etc.) could be made as tubes in different sizes, for example, a 25 mm “double D” configuration with 7 mm lumen or a 10 mm long tube with 7 mm lumen. A PVA sponge in a dried state is easily compressed and could fully re-hydrate and expand to its original state in minutes. One aspect of the invention is to introduce PVA sponge tube with optimal porosity in a compressed dry form as an endo-plug and allow it to expand in-situ across aneurysm. Through lumen of the tube would then be stented or stent-grafted at a diameter less than that of the expanded sponge. The porous sponge plug could be compressed by applying vacuum, by wrapping or injected in a funnel. The dried sponge plug could be crimped on a stent or balloon, pushed through sheath over a wire, or premounted on its own delivery apparatus.

The delivery sheath method comprises a first step of inserting a long sheath with a tip marker up to the insertion site in a patient. Load the compressed plug on a pusher/cannula and then insert the plug/cannula through sheath up to a desired deployment site. After deployment, withdraw sheath until cannula marker and sheath tip marker line up. This will anchor the distal about one cm of sponge in sheath while majority of the sponge is hydrated. Thereafter, complete deployment by withdrawing sheath over the distal one cm to release the sponge in place.

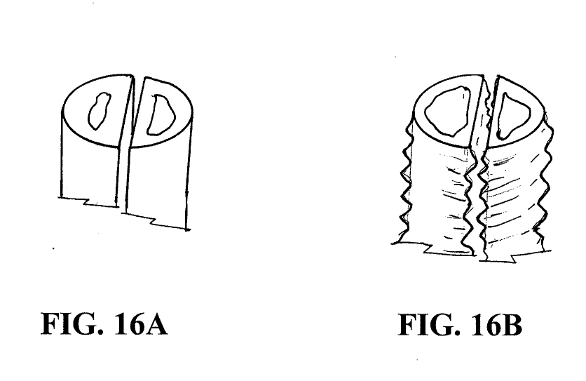

FIG. 16A shows “double D” sponges and FIG. 16B shows “ribbed sponges” to provide interlocked seal in blood vessels. The conformable pair of sponges allows insertion of bifurcated grafts in 2 parts from each groin, resulting in lower profiles. In one embodiment, the delivery cannula has multiple hydration holes to speed expansion of sponge. In another embodiment, pulse delivery of warm saline speeds sponge expansion too.

One aspect of the invention provides a conformable pair of spongy endo-plugs for treatment of aneurysmal vessels, wherein the plugs are compressed in a first configuration for delivery to the vessels and expanded via re-hydration to a second configuration to plug the vessels. In one embodiment, the plug has a through lumen. In another embodiment, each plug has matching flat surface facing each other. In still another embodiment, each plug has a matching ribbed surface to provide interlocked seal in vessels. In an alternate embodiment, the expansion of the endo-plug is enhanced with a shape memory Nitinol wire.

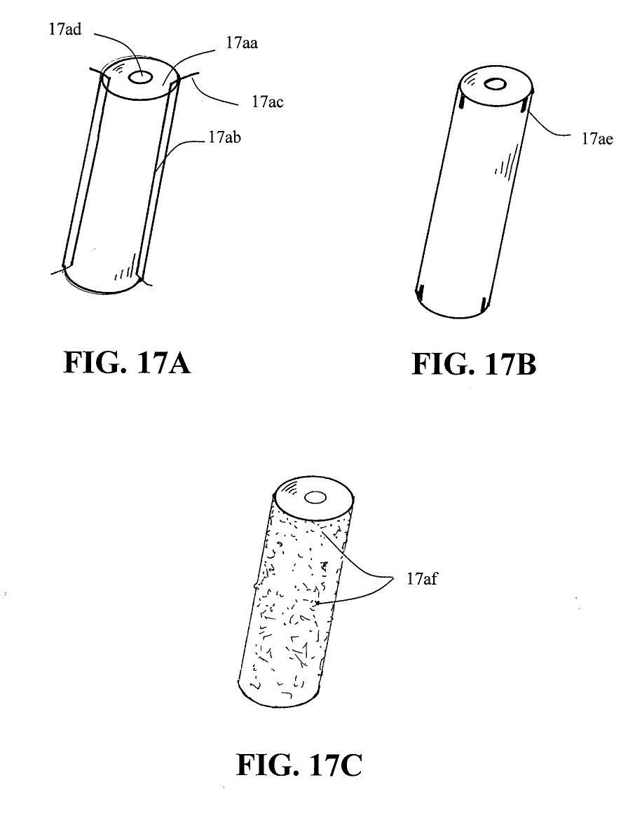

The sponge plug (17aa) can be reinforced or supported with anchor structures as shown in FIG. 17A. The sponge plug has an embedded wire struts (17ab), hooks (17ac) and a through lumen (17ad). The sponge plus can also incorporate radiopaque elements as markers (17ae) or tantalum powder (17af) for x-ray visualization (FIG. 17B and FIG. 17C).

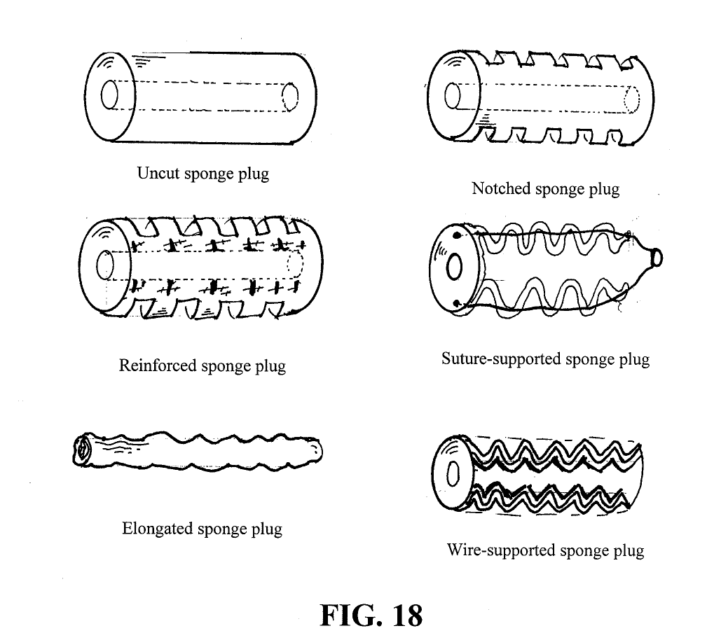

FIG. 18 shows various configurations of sponge endo-plugs, including the suture-supported sponge plug that can change the shape by tightening the suture and the wire-supported sponge plug that can change the shape when the wire is made of shape-memory Nitinol material or the like.

One aspect of the invention provides a spongy endo-plug for treatment of aneurysmal vessels, comprising an anchoring means for securing the plug in place without undue migration. In another embodiment, the endo-plug is configured radiopaque or incorporated with at least one radiopaque market.

### The Endoleak

Exclusion of the aneurysm sac is the main goal of the stent-graft treatment, and clinical success is defined by the “total exclusion” of the aneurysm. However, at times, failure of the stent-graft to totally exclude blood flow to the aneurysm sac may occur. As a matter of fact, endoleak is the major cause of complications, and thus failure in endoluminal treatment of AAA. Endoleak is a term that describes the presence of persistent flow of blood into the aneurysm sac after device placement. The management of some types of endoleak remains controversial, although most can be successfully occluded with surgery, further stent implantation, or embolization. Four types of endoleaks have been defined, based upon their proposed etiology.

A type I endoleak, which occurs in 0 to 10 percent of endovascular aortic aneurysm repairs, is due to an incompetent seal at either the proximal or distal attachment site. Etiologies include undersizing of the diameter of the endograft at the attachment site and ineffective attachment to a vessel wall that is heavily calcified or surrounded by thick thrombus. Although such a leak can occur immediately after placement, a delayed type I endoleak may be seen on follow-up studies if the device is deployed into a diseased segment of aorta that dilates over time, leading to a breach in the seal at the attachment site.

Type I endoleaks must be repaired as soon as they are discovered because the aneurysm sac remains exposed to systemic pressure, predisposing to aneurysmal rupture, and spontaneous closure of the leak is rare. If discovered at the time of initial placement, repair may consist of reversal of anticoagulation and reinflation of the deployment balloon for an extended period of time. These leaks may also be repaired with small extension grafts that are placed over the affected end. These methods are usually sufficient to exclude the aneurysm. Conversion to an open surgical repair may be needed in the rare situation in which the leak is refractory to percutaneous treatment.

Type II endoleaks are the most prevalent type, occurring in 10 to 25 percent of endovascular aortic aneurysm repairs, and describe flow into and out of the aneurysm sac from patent branch vessels. They are most often identified on the postprocedural CT, appearing as collections of contrast outside of the endograft, but within the aneurysm sac. The most frequent sources of type II endoleaks are collateral back flow through patent lumbar arteries and a patent inferior mesenteric artery. Because the sac fills through a collateral network, the endoleak may not be visualized on the arterial phase of CT scanning; thus, delayed imaging is required.

The significance and management of type II endoleaks is controversial. Some investigators argue that, since spontaneous resolution occurs in 30 to 100 percent of cases, a “wait and see” approach is preferable, while carefully following aneurysm volume and morphology on CT imaging. However, systemic pressures have been noted within the aneurysm sac in the presence of type II endoleaks, indicating a more tenuous situation.

Type III and type IV endoleaks are much less common. Type III endoleaks represent flow into the aneurysm sac from separation between components of a modular system, or tears in the endograft fabric. Type IV endoleaks are due to egress of blood through the pores in the fabric. Type IV leaks heal spontaneously, while type III leaks are repaired with an additional endograft to eliminate systemic flow and pressure in the aneurysm.

Flow identified within the aneurysm sac (endoleaks) can represent a failure of the attachment sites (type I) or device (type III). There is general agreement that these failure modes necessitate urgent repair because blood flow and systemic pressure will continue to be transmitted into the aneurysm sac, putting the patient at continued risk for aneurysm enlargement and rupture.

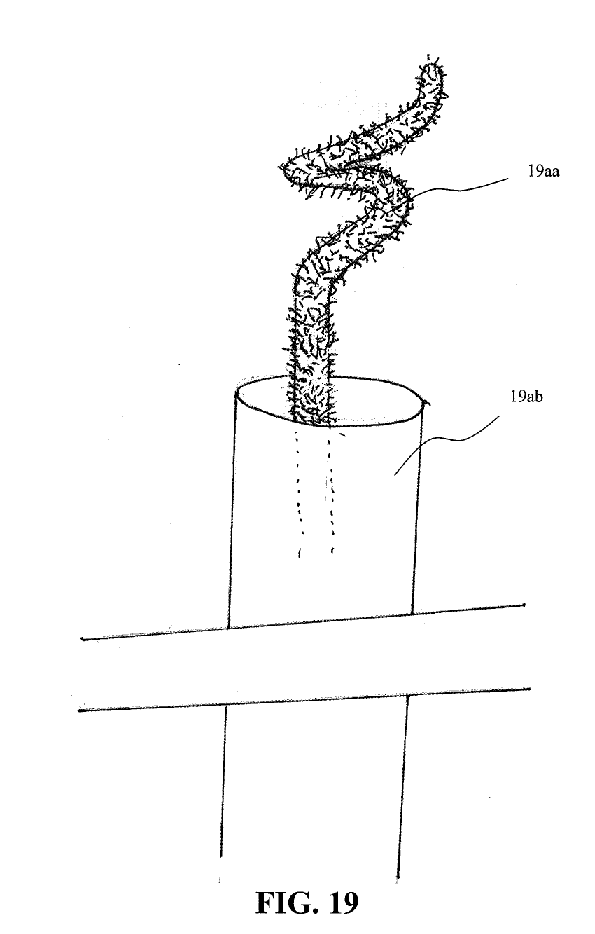

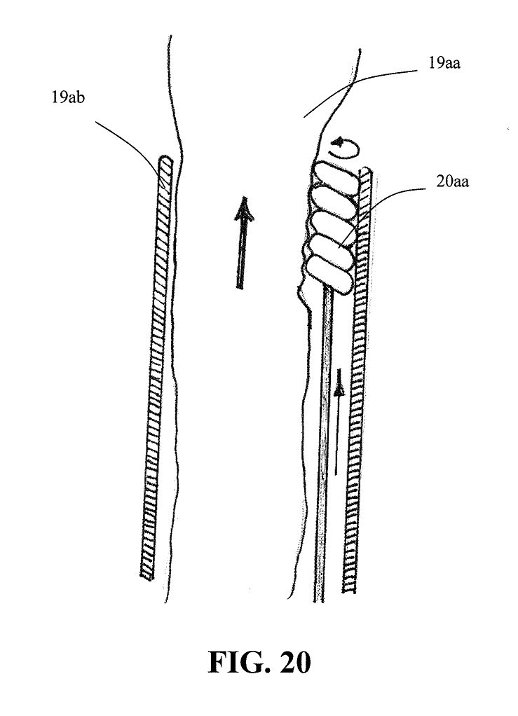

One aspect of the invention relates to devices and methods for endoleak solutions by extruding or inserting soft, thrombogenic ‘pipe-cleaner’ like soft filler material (19aa) into AAA sac, preferably through a delivery catheter (shown in FIG. 19). The material could be PVA (polyvinyl alcohol), Dacron (polyester) thread and the like with enhanced thrombogenic properties. The diameter of the ‘pipe-cleaner’ like material could be from thread-like (0, 0-0) up to 10-20 mm. Since the material is soft and cannot be pushed, one solution is to pull the ‘pipe-cleaner’ like material through a catheter (19ab) by a tip mechanism (as shown in FIG. 20). In one embodiment, the tip (20aa) is configured to move helically forward when turned in one direction so to pull the material outwardly. The turning of the tip can be either via a connected mandrill or wire that transmits the torque to a proximal handle of the catheter (19ab), or via saline injection to push and turn the tip section. After the material is placed inside the sac and separated from the tip, the tip is withdrawn into the catheter lumen when the tip is turned in an opposite direction. And the catheter is withdrawn from the patient.

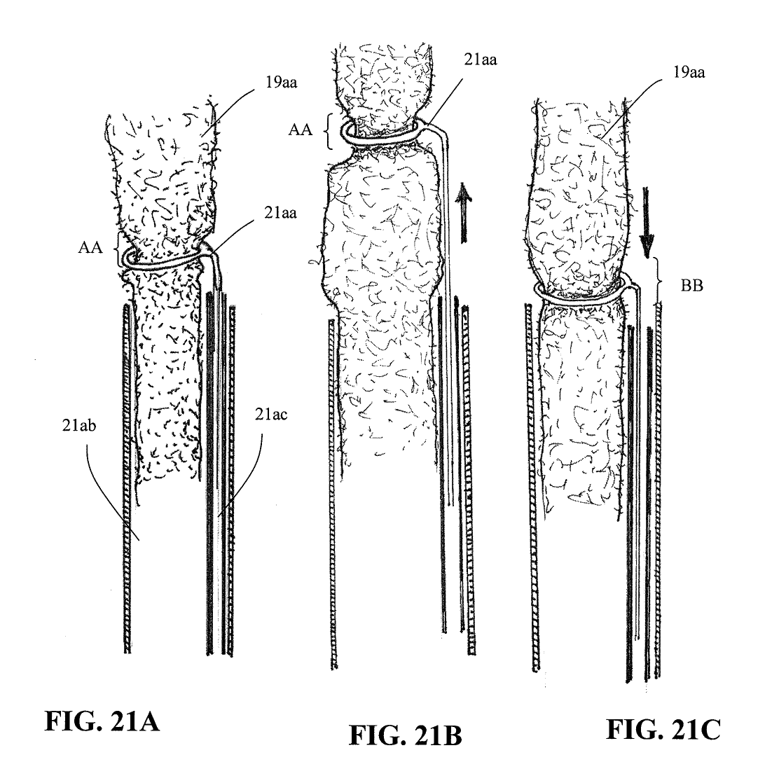

In another embodiment, the soft filler material as shown in FIG. 19 may be pulled out of a catheter by a repositionable snare that may movably be located in a second lumen (21ac) of a dual-lumen catheter. FIG. 21A shows a snare (21aa) engaged with the soft filler material at point AA in a first lumen (21ab) of a dual-lumen catheter, whereas FIG. 21B shows the soft filler material (19aa) is pulled upward by the snare. The snare is thereafter loosened from the soft filler material at point AA and repositioned at point BB and engaged with the soft filler material again (shown in FIG. 21C) so to repeat the engagement-pulling-disengagement-reposition operation until the soft filler material (19aa) is inserted into the sac as desired.

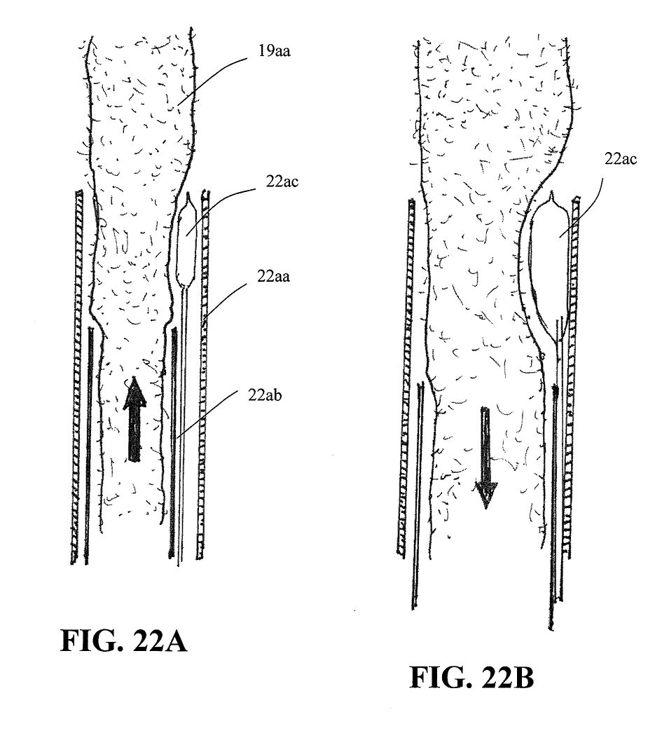

In an alternate embodiment, a catheter set with a concentric inner catheter (22ab) and an outer catheter (22aa) is used to deliver the soft filler material (19aa) into the sac, wherein a balloon (22ac) is movably located inside the gap between the lumen of the outer catheter and the sheath of the inner catheter. In one embodiment, the balloon is sized and configured to show a circumferential concave surface. The soft filler material occupies the lumen of the inner catheter tightly and/or intimately before the insertion step. The catheter set is then delivered to the sac region. In operations, the inner catheter is first pushed outwardly to deliver part of the soft filler material inside the sac as shown in FIG. 22A. The distal end of the inner catheter is pushed outwardly and engages the balloon at about the proximal edge of the balloon. Then the balloon (22ac) is inflated to pin the soft filler material against the sheath of the outer catheter so the inner catheter can be retracted inwardly. The operation can be repeated until all soft filler material is delivered inside the sac.

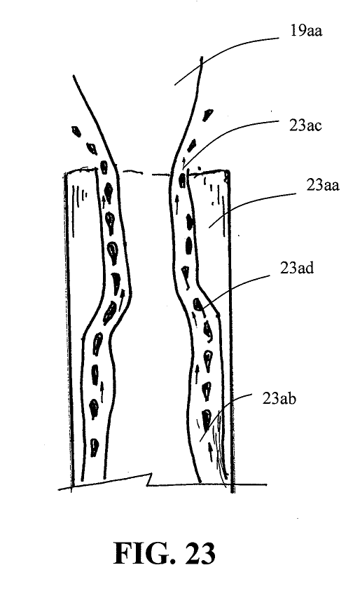

In still another embodiment, a nozzle catheter with a narrowed distal section can be used to hydraulically deliver the soft filler material into the sac. FIG. 23 shows a nozzle catheter (23aa) of the present invention, comprising a catheter lumen (23ab), a necked-down lumen (23ac), wherein the soft filler material occupies a portion of the catheter lumen in a loose manner. Saline or appropriate fluid (23ad) is hydraulically introduced at a speed substantially to squeeze the soft filler material through the necked-down section so to push or carry the soft material into the sac.

Some aspects of the invention relate to a method of inserting soft, thrombogenic ‘pipe-cleaner’ like soft filler material (19aa) into AAA sac, preferably through a delivery catheter. The material could be made of PVA (polyvinyl alcohol), Dacron (polyester) thread and the like with enhanced thrombogenic properties.

### AAA Device and Methods

Some aspects of the invention relate to an improved modular AAA device that meets clinical needs of a percutaneous delivery (preferably with a 12 French or smaller delivery catheter) in a cath-lab with local anesthesia. The modular device may have multiple sizes, but not custom-made. The device is configured fully adaptable anatomically with respect to neck attachment, tortuosity and iliac anatomy, among others. The current device is particularly suitable for implantation in a patient with a short neck and/or two renal arteries not at the same axial elevation along the aorta. FIG. 7 shows some procedures and means for solving the problems of two renal arteries not at the same axial elevation along the aorta. FIG. 9 shows some procedures and means for solving the problems of a short neck.

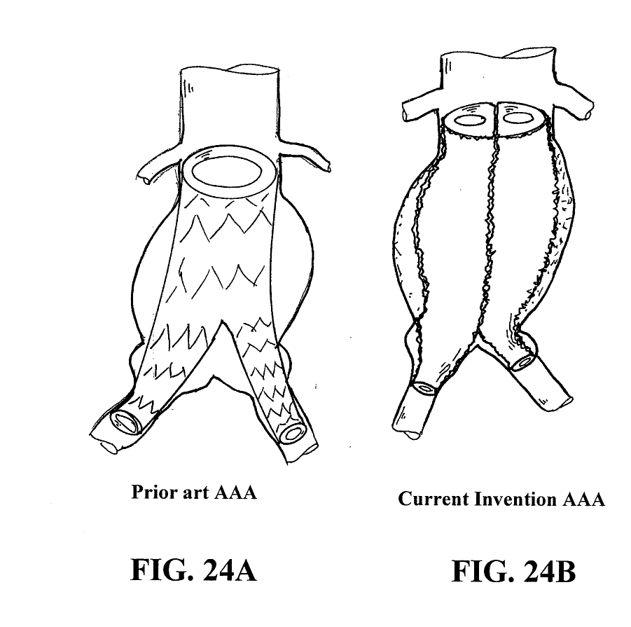

FIG. 24 shows comparison of: (A) a conventional AAA device, and (B) an improved AAA device of the present invention. The prior art device is usually a tubular graft with a bifurcated distal section for inserting into iliac arteries. The limitations of conventional devices may include, among others, large introducer size, metal/fabric construction, prone to endoleak, need for exact size, and need for large device inventory. The new improved device of the present invention may comprise: compressible foam tube, percutaneous delivery, 2-10 mm lumens, introduced soft material, cured in-situ with UV, heat or chemical reaction, and lattice of foam filling blood vessels.

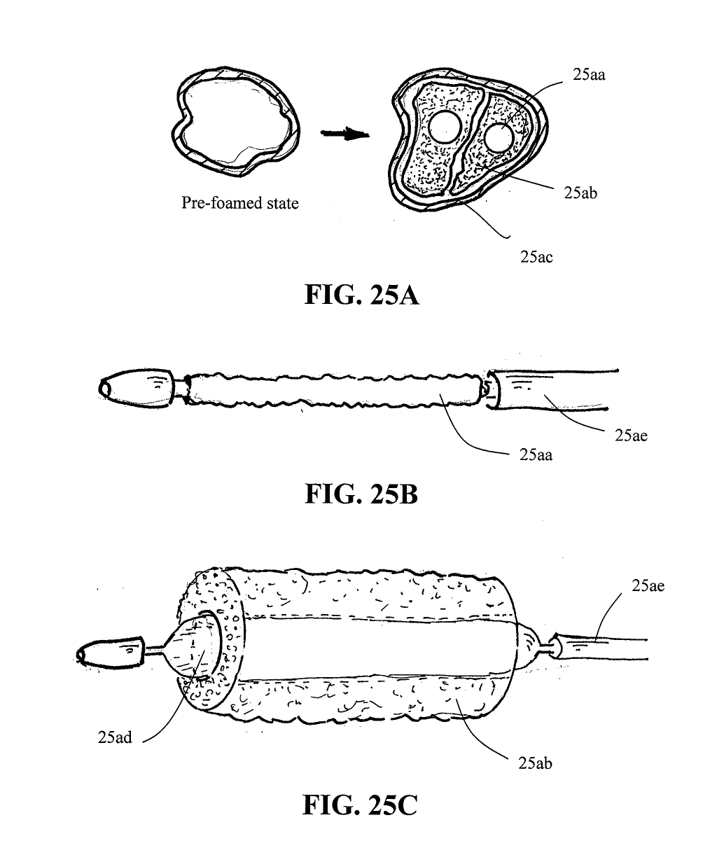

As foam cures, it becomes harder which relieves pulsatile wall stress on aneurysm (25ac) in-situ. In initial soft configuration, foam (25ab) fills lumen to seal (as shown in FIG. 25A). The foam tube was introduced in compressed configuration (as shown in FIG. 25B) over a balloon (25ad) or other expandable means (stents, basket, etc.) from a delivery apparatus (25ae) for expanding the compressed foam tube (25aa). The foam tube expands with fluid contact and/or balloon expansion (as shown in FIG. 25C). The foam lattice becomes hardened by curing with UV, heat, chemical or biological via balloon delivery. The curing time could be from about 1 minute to weeks depending on material selection to meet clinical needs.

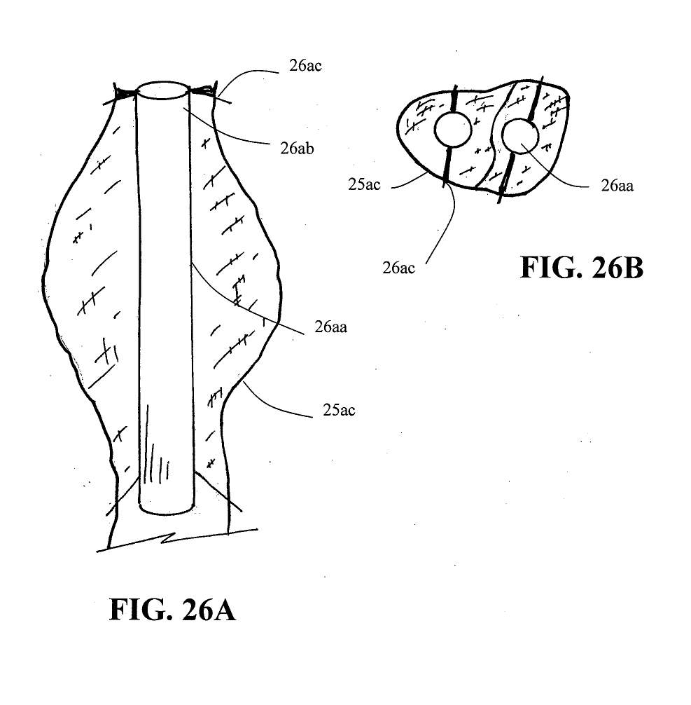

In one embodiment, the tubular graft (26aa) comprises cuffs (26ab) at each end, wherein the cuff has prongs (26ac) that hold the graft in place while the cuffs heal (as shown in FIG. 26A). FIG. 26B shows a top cross-sectional view of the tubular graft (26aa). In another embodiment, the cuff of the endograft system of the present invention comprises a foam cuff, wherein the foam may be made from hardenable foam material and hardened in situ. In still another embodiment, the first proximal end of a first endograft is at a substantial distance proximal to the second proximal end of a second endograft.

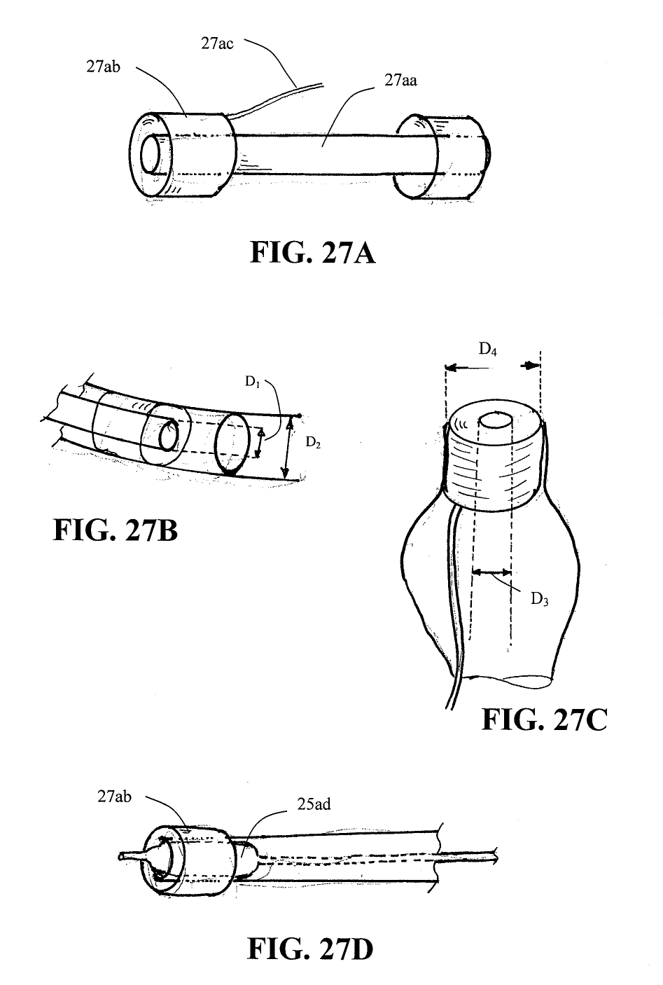

In another embodiment, a device for creation of a low-profile, percutaneous delivery, endoleak resistant vascular graft is shown in FIG. 27A. The principal concept for such a device (27aa) is an inflatable prosthesis, preferably with inflatable ends (27ab) and/or an inflation body (27ac), with a through lumen. The prosthesis solves the two major drawbacks of prior art stent-grafts of: a large introduction size and difficult vessel sizing resulting in endoleaks. The prosthesis could be introduced in a compressed form and inflated with a fluid (for example, contrast and/or saline) to position and test for leaks. When properly positioned the cuffs would be deflated and reinflated with a liquid polymer which would set and harden. The hardenable liquid polymer may include EVAL (poly(ethylene-co-vinyl alcohol)), cellulose acetate, p-HEMA (poly(2-hydroxyethyl methacrylate)), acrylates, combinations thereof, and the like). The prosthesis would be made of an ultrathin microporous material such as PTFE, polyester and the like. Each layer would be very thin (for example, less than 50 mμ) to reduce the compressed profile. p-HEMA is a polymer that forms a hydrogel in water. p-HEMA functions as a hydrogel by rotating around its central carbon. In air, the non-polar methyl side turns outward, making the material brittle and easy to grind into the correct lens shape. In water, the polar hydroxyethyl side turns outward and the material becomes flexible.

The cuffs (27ab) could be sized and configured to be minimally larger than a graft for use in smaller vessels (as shown in FIG. 27B) or significantly larger in vessels such as in the aorta (as shown in FIG. 27C). For example, the lumen diameter, D1, could be between about 2 and 10 mm whereas the cuff outer diameter, D2, could be between about 4 and 12 mm. Preferably in another application, the lumen diameter, D3, could be between about 6 and 14 mm whereas the cuff outer diameter, D4, could be between about 24 to 36 mm.

The cuffs could be introduced separately or they could be an integral part pf endograft. FIG. 27D shows that the cuffs and/or graft could be temporarily fixed in place during the inflation and positioning phase by placement over an angioplasty balloon (25ad).

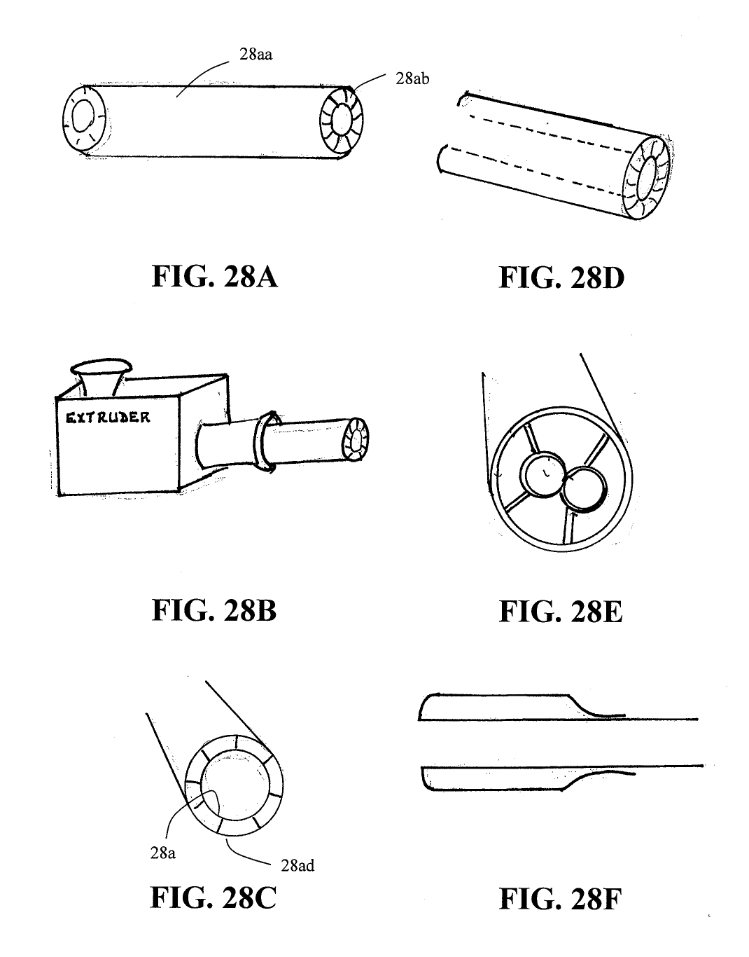

In another embodiment, a double-walled, baffled tube filled with a hardening or form-filling material would function as a flexible graft with sufficient hoop strength to obviate the use of another support structure such as a metallic stent. The baffles (28ab) of the tube graft (28aa) are filled with liquid, self-hardening polymer (as shown in FIG. 28A). In one embodiment, the baffles only extend from an edge of the tube graft inwardly for a proper short distance (toward the opposite end) configured to provide adequate hoop retention strength. One method of baffle tube construction could be extrusion of PTFE in a 2 layer, single or multi-lumen configuration with supporting baffles as shown in FIGS. 28B and 28C. Both inner layer (28ac) and outer layer (28ad) have a wall thickness of about 10 to 30 microns. After extrusion the ends are sealed to create what is essentially a balloon with a through lumen (as shown in FIG. 28D). It is also useful to have 2-lumen extrusion with baffles (as shown in FIG. 28E). In an alternate embodiment, a portion of the baffled layer of at least one end of a tube could be everted to create a cuff (as shown in FIG. 28F).

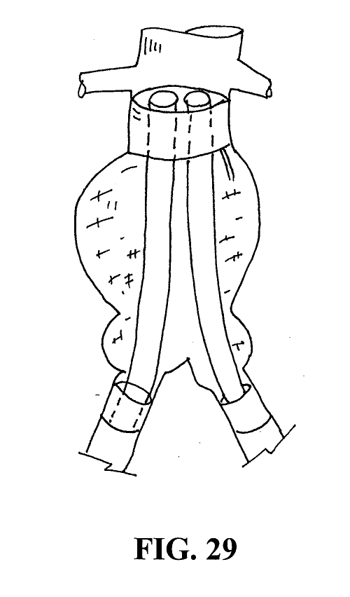

In a separate embodiment, the cuffs can be constructed with multiple through lumens so that bifurcated channels can be formed (see FIG. 29). In this fashion, the aorta can be occluded using a low profile, “2-hole” cuff and two small diameter grafts. For example, two 10 mm stent grafts can be inserted percutaneously, whereas a single 24 mm graft cannot (prior art).

### Introduction Methods for In-situ Foam Grafts

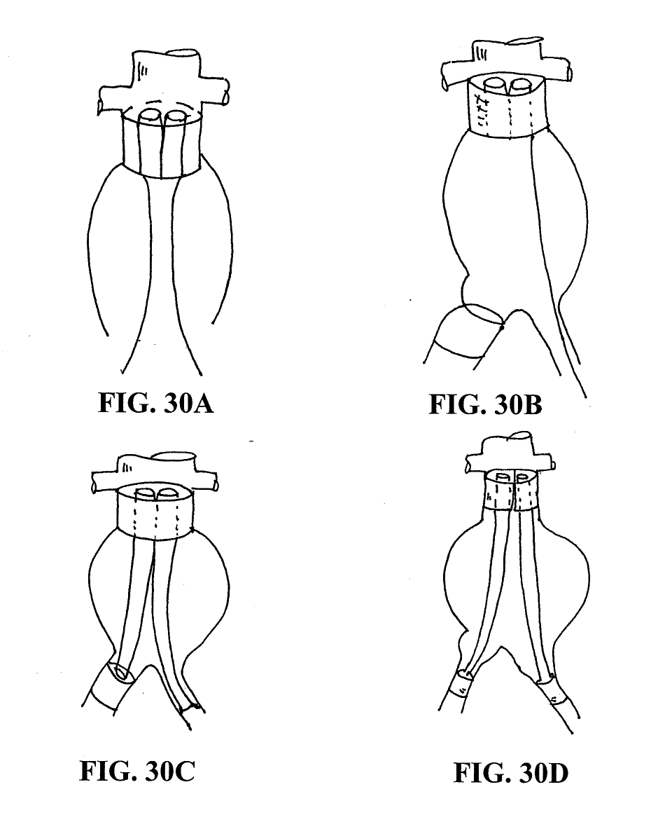

After a first cuff is introduced into and occupy the aortic region below the renal arteries (as shown in FIG. 30A), introduce a balloon catheter into the first cuff and inflate the balloon (step 1). Using a microcatheter or other appropriate means in cuff lumen to inflate cuff with fluid (step 2). Then, catheterize a second lumen at the first cuff region (step 3). Using angiogram to check position and seal; reposition if necessary (step 4). FIG. 30B shows steps of inserting a second cuff in iliac artery in a reduced diameter manner (step 5). Fill cuffs with liquid polymer and cure the liquid polymer using heat, UV, solvent dissolution, chemical reaction or precipitation (step 6). Then insert stent-grafts as shown in FIG. 30C (step 7). In an alternate embodiment, a 2-cuff graft with conformable or D-shaped cuffs (as shown in FIG. 30D) could be applied.

### Balloon Endograft

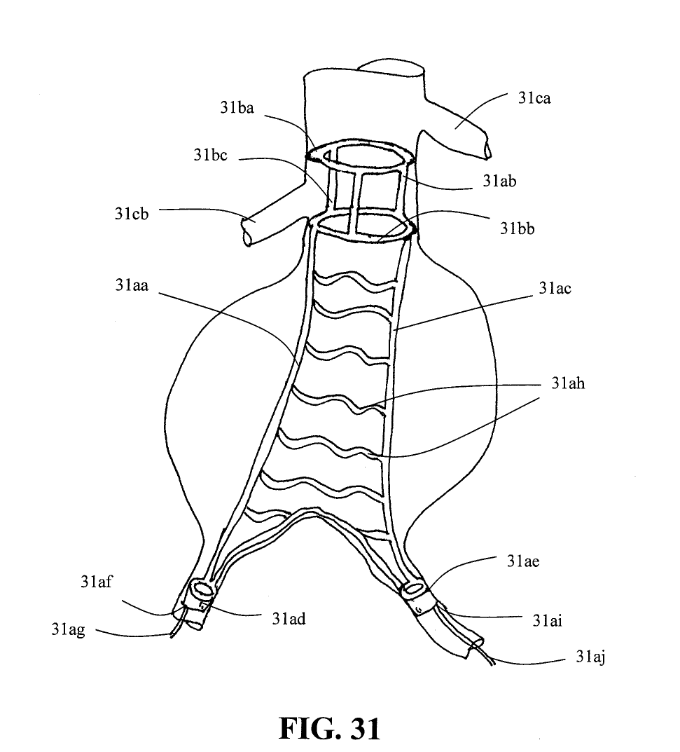

FIG. 31 shows one embodiment of an endograft made of double layer inflatable balloon without metal or rigid supporting component (“balloon endograft”). The balloon endograft (31aa) is made of double layers with a space between the double layers, wherein the space is inflatable with fluid, saline or hardenable soft polymer. In one embodiment, the endograft (31aa) comprises a neck attachment member (31ab), a tubular main body (31ac) and bifurcated distal ends (31ad, 31ae), wherein the neck attachment member may comprise an upper neck attachment ring unit (31ba), a lower neck attachment ring unit (31bb) and at least two connecting units (31bc) that connect the upper and lower neck attachment ring units with throughput lumen for fluid communication. In one preferred embodiment, the upper neck attachment ring unit (31ba) is configured to be placed between the proximal renal artery (31ca) and the distal renal artery (31cb) whereas the lower neck attachment ring unit (31bb) is configured to be placed distal to the distal renal artery (31cb). In another preferred embodiment, the number of connecting units (31bc) is three or more so to maintain the two neck attachment ring units substantially parallel to each other. In one embodiment, there provides an optional introduction port at one or both distal ends, wherein the introduction port is self-sealing or with a one-way valve for infusing fluid into the space to inflate the inflatable endograft.

In one exemplary embodiment, the balloon endograft is collapsed for delivery via a delivery sheath or catheter to the AAA site with a minimum profile. Once the neck attachment member is placed at about the renal artery ostia and the two bifurcated distal ends are placed in the right and left iliac arteries respectively, fluid or hardenable polymer foam is introduced through the first introduction port (31af) via an infusing catheter (31ag). The hardenable polymer foam is infused until the space is totally filled with the foam, followed by curing or hardening in situ. In one preferred embodiment, the upper and lower neck attachment ring units are securely anchored to the aorta walls once the neck attachment member is inflated.

In an alternate embodiment, the balloon endograft is configured to have corrugated configuration (31ah). The corrugation with internal space is in fluid communication with the second introduction port (31ai). The hardenable polymer foam may be introduced through the second introduction port (31ai) via an infusing catheter (31aj) to fill the corrugation space (31ah). The corrugation of the balloon endograft is sized and configured to support and reinforce the endograft against endoleak. Some aspects of the invention relate to a balloon endograft (without any metallic or rigid supporting members before deployment) comprising: a neck attachment member, a body and two bifurcated distal ends, wherein the endograft is with double layers and a space between the layers, the space is configured to be filled with fluid or hardenable foam to inflate the balloon endograft. In one embodiment, the body is configured in a corrugated configuration. In another embodiment, the body serves to direct blood flow bypassing the aneurysm.

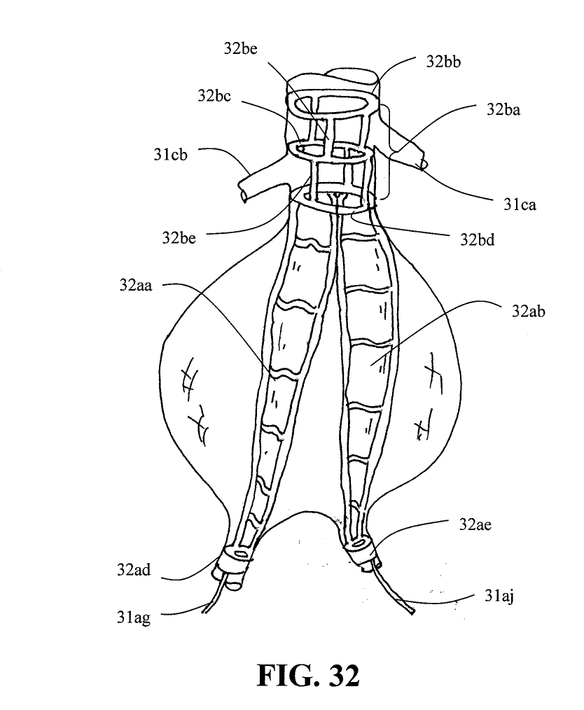

FIG. 32 shows one embodiment of an endograft made of two double layer inflatable balloon bodies without metal or rigid/stiff supporting component (“balloon endograft”). The balloon endograft having two individual graft bodies (32aa, 32ab) is made of double layers with a space between the double layers, wherein the space is filled with inflatable fluid, saline or hardenable soft polymer. In one embodiment, the endograft comprises a neck attachment member (32ba), two tubular main bodies (32aa, 32ab) with their respective distal ends (32ad, 32ae), wherein the neck attachment member may comprise an upper neck attachment ring (32bb), a middle neck attachment ring (32bc), and a lower neck attachment ring (32bd) and at least two connecting units (32be) that connect the upper to middle rings or middle to lower neck attachment rings with throughput lumen for fluid communication. In one preferred embodiment, the upper neck attachment ring (32bb) is configured to be inflated and securely positioned proximal to the upper renal artery (31ca). The middle neck attachment ring (32bc) is configured to be placed between the proximal renal artery (31ca) and the distal renal artery (31cb) whereas the lower neck attachment ring unit (32bd) is configured to be placed distal to the distal renal artery (31cb). In another preferred embodiment, the number of connecting units (32be) is three or more so to maintain the neck attachment rings substantially spaced apart and parallel to each other. In one embodiment, there provides an optional introduction port at one or both distal ends, wherein the introduction port is self-sealing or with a one-way valve for infusing fluid into the space to inflate the inflatable endograft.

Some aspects of the invention relate to a balloon endograft comprising: a neck attachment member, a body and at least one distal end, wherein the endograft comprises double layers and a space between the layers, the space being configured to be filled with inflatable fluid or hardenable foam to inflate the balloon endograft. In one embodiment, the endograft is characterized with no stiff or rigid supporting component prior to inflating the balloon endograft. In another embodiment, the body comprises two inflatable tubes, each inflatable tube having a proximal end secured to the neck attachment member, a distal end, and double layers with a space between the layers. In still another embodiment, the graft body is configured in a corrugated configuration to enhance hoop strength and prevent the graft body from collapsing. In a preferred embodiment, the neck attachment member comprises two inflatable neck attachment rings and at least two connecting units that connect the two rings, wherein the neck attachment rings are inflatable to anchor securely at wall of a blood vessel.

From the foregoing, it should now be appreciated that a device system for treating abdominal aortic aneurysms has been disclosed. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

What is claimed is:

1. An endograft system for treatment of an abdominal aortic aneurysm (AAA), comprising a cuff and at least two endograft units, each endograft unit having a lumen, a proximal end and a distal end, wherein said endograft units are made of flexible water-tight tubes having the proximal ends placed and secured at the cuff and the distal ends to be placed and fixed in each of iliac arteries.

2. The endograft system according to claim 1, wherein said endograft units are made of compressible water-tight foam tubes.

3. The endograft system according to claim 1, said system comprising four endograft units, each endograft unit having a lumen, a proximal end and a distal end, all four proximal ends are placed and secured at the cuff whereas a first distal end extends and is to be fixed in right iliac artery, a second distal end extends and is to be fixed in left iliac artery, a third distal end extends and is to be fixed in right renal artery and a fourth distal end extends and is to be fixed in left renal artery.

4. The endograft system according to claim 1, wherein the cuff has prongs that hold the endograft units in place.

5. The endograft system according to claim 1, wherein the cuff comprises a foam cuff.

6. The endograft system according to claim 5, wherein the foam is made of hardenable foam material.

7. The endograft system according to claim 6, wherein the foam material is selected from the group consisting of polyvinyl alcohol foam, poly(ethylene-co-vinyl alcohol), cellulose acetate, poly(2-hydroxyethyl methacrylate), acrylates, and combinations thereof.

8. The endograft system according to claim 6, wherein the foam material is treated with UV light or heat.

9. The endograft system according to claim 1, wherein the first proximal end of a first endograft unit is located at a substantial distance proximal to the second proximal end of a second endograft unit.

10. The endograft system according to claim 1, wherein the endograft unit comprises an inner layer of a water-tight flexible tube, a middle layer of semi-rigid mesh-like material, and an outer layer of water-tight flexible overlap, wherein the endograft unit is characterized with at least two water-tight layers.

11. The endograft system according to claim 10, wherein the inner layer is made of stretchable PTFE tube and the outer layer is made of stretchable PTFE overlap.

12. The endograft system according to claim 1, wherein the endograft unit is compressible and expandable.

13. The endograft system according to claim 1, wherein said endograft units are made of microfiber woven material.

14. A method for treatment of AAA using an endograft system according to claim 1, the method comprising steps of: (a) placing a renal stent inside a renal artery, wherein a first end of the renal stent is inside the renal artery and wherein a second end of said renal stent is positioned at about the renal artery ostium; (b) placing a first endograft unit in the AAA area, wherein said endograft unit intimately and compressively contacts the renal artery ostium; (c) providing a wire from inside the lumen of said endograft unit at about the ostium site and piercing through said endograft unit so to create a hole facing the renal artery configured for blood communication from aorta to the renal artery.

15. The method according to claim 14, further comprising a step of balloon expansion at about the hole to enlarge the hole and urge the second end of the renal stent to protrude into said endograft unit.

16. The method according to claim 14, further comprising steps of: (a) placing a second renal stent inside a second renal artery, wherein a first end of the second renal stent is inside the second renal artery and wherein a second end of said second renal stent is positioned at about the second renal artery ostium; (b) placing a second endograft unit in the AAA area, wherein said second endograft unit intimately and compressively contacts the second renal artery ostium; (c) providing a wire from inside the lumen of said second endograft unit at about said second ostium site and piercing through said second endograft unit so to create a hole facing the second renal artery configured for blood communication from aorta to the second renal artery.

17. The method according to claim 14, wherein the wire is provided via a guide catheter from outside of a patient.

18. A method for treatment of AAA using an endograft system according to claim 1, the method comprising steps of: (a) placing a renal stent inside a renal artery, wherein a first end of the renal stent is inside the renal artery and wherein a second end of the renal stent protrudes beyond a renal artery ostium; (b) placing the endograft unit in about the AAA area, wherein the endograft unit intimately contacts the renal artery ostium; (c) applying RF energy to the second end of the renal stent so to create a hole on the endograft unit and to cause the renal stent to protrude into the lumen of the endograft unit configured for blood communication from aorta to the renal artery.

19. A method for treatment of circumscribed dilatation of a large blood vessel, comprising positioning a modular multi-luminal endograft system for reducing a diameter of said large blood vessel by generating multiple lumina for down-stream flow continuity.

20. An endograft for treatment of an abdominal aortic aneurysm (AAA) comprising a neck attachment section, a graft body, and a leg section, the neck attachment section having a multiple-anchoring mechanism that comprises at least a first anchoring element for placement at proximal to a renal artery and a second anchoring element axially spaced apart from the first anchoring element, wherein the second anchoring element is configured for placement at distal to said renal artery.

21. The endograft according to claim 20, wherein the multiple-anchoring mechanism comprises a third anchoring element configured for placement at about a region between two renal arteries.

22. An endograft for treatment of AAA comprising a neck attachment section, a first foam tube having a proximal end and a length to extend from the neck attachment section to a first iliac artery for fixation inside the first iliac artery, and a second form tube having a proximal end and a length to extend from the neck attachment section to a second iliac artery for fixation inside the second iliac artery, wherein both foam tubes are secured to the neck attachment section.

23. The endograft according to claim 22, wherein the first proximal end of a first foam tube is located at a substantial distance proximal to the second proximal end of a second foam tube.

24. The endograft according to claim 22, wherein the neck attachment element comprises a hanger, and wherein the proximal end of the first foam tube is configured with a hook to securely couple the hook to the hanger.

25. The endograft according to claim 22, wherein the proximal end of the first foam tube is magnetically coupled to the neck attachment element.

26. The endograft according to claim 22, wherein a distal end of the first foam tube is flared to anchor and seal the distal end to surrounding tissue of the first iliac artery.

27. The endograft according to claim 22, wherein a distal end of the first foam tube is balloon expanded to anchor and seal the distal end to surrounding tissue of the first iliac artery.

28. The endograft according to claim 22, wherein a distal end of the first foam tube is made of shape memory material to anchor and seal the distal end to surrounding tissue of the first iliac artery.

29. The endograft according to claim 22, wherein a proximal section of said foam tubes is made of inflatable elements, and wherein said proximal section is distendable to anchor and secure the proximal section against wall of a blood vessel.

30. The endograft according to claim 22, wherein at least one of the foam tubes further comprises an inflatable tube body.

31. The endograft according to claim 22, wherein at least one of the foam tubes comprises a double-walled, baffled tube body filled with form-filling material that functions as a flexible graft with sufficient hoop strength to obviate use of a radial positioning structure.

32. The endograft according to claim 31, wherein a portion of the baffled layer of at least one end of the foam tube is everted to create a cuff.

33. The endograft according to claim 22, wherein an aneurysm sac of the AAA is filled with foam material that is subsequently hardened in situ.

34. The endograft according to claim 33, wherein the foam material is introduced via a one-way valve mounted on the first form tube into the aneurysm sac.

35. The endograft according to claim 33, wherein the foam material is selected from the group consisting of polyvinyl alcohol foam, poly(ethylene-co-vinyl alcohol), cellulose acetate, poly(2-hydroxyethyl methacrylate), acrylates, and combinations thereof.

36. The endograft according to claim 33, wherein the foam material is treated with UV light or heat in situ.

37. A flexible stent graft for inserting into a blood vessel, comprising a distal section, a proximal section and a graft body with a lumen that connects the distal and proximal sections, said graft having a first layer of flexible rigid or semi-rigid material, and a second layer of water-tight flexible overlap, wherein the graft is collapsible and is characterized with a low profile during the inserting operation.

38. The stent graft according to claim 37, wherein the first layer comprises a spiral wire that is compressible within a sheath during the inserting operation.

39. The stent graft according to claim 37, wherein the second layer invaginates onto the first layer after the first layer is positioned in place.

40. The stent graft according to claim 37, further comprising a third layer of water-tight flexible tube, wherein the graft is characterized with at least two water-tight layers.

41. The stent graft according to claim 37, wherein the third layer is made of stretchable PTFE tube and the second layer is made of stretchable PTFE overlap.

42. The stent graft according to claim 37, wherein the proximal section is shaped in a D-shaped configuration.

43. The stent graft according to claim 37, wherein a sleeve at an end of the stent graft is formed by inverting an extra length of the third layer over the first and second layers.

44. The graft according to claim 43, wherein the inverted sheath is secured to the first layer by fastening means for securing the inverted sheath with the first layer, said fastening means comprising suturing, stapling, gluing, or bonding.

45. The stent graft according to claim 37, wherein the third layer is made of flexible fabrics or polymer tube and the second layer is made of flexible fabrics or polymer overlap.

46. The stent graft according to claim 45, wherein the second layer or the third layer is made of substantially water-tight microfibers woven material.

47. The stent graft according to claim 37, wherein barbs are incorporated and spaced apart appropriately at about the proximal section of the stent graft configured for anchoring said graft at wall of a blood vessel.

48. The stent graft according to claim 47, wherein the barbs are made of shape memory material or temperature-sensitive material.

49. The stent graft according to claim 37, wherein anchors are provided at about the proximal section of said graft configured for anchoring said graft at wall of a blood vessel as a secondary operation.

50. A stent graft system comprising a first and a second stent grafts of claim 4, wherein the proximal section of either stent graft is shaped to have a semi-circular like side and a mating side, wherein the first mating side of the first stent graft mates and matches intimately the second mating side of the second stent graft when the proximal sections of the two grafts are mated against each other to form a cylindrical-like tubular configuration.

51. The stent graft system according to claim 50, wherein the first distal section of the first stent graft is flexible for inserting into a right iliac artery and the second distal section of the second stent graft is flexible for inserting into a left iliac artery.

52. The stent graft system according to claim 50, wherein the first mating side of the first stent graft is configured to have positive charged magnet and the opposite second mating side of the second stent graft is configured to have negative charged magnet so to ensure control seal and intimate contact upon been mated.

53. The stent graft system according to claim 50, wherein the proximal sections of the two stent grafts in the cylindrical-like tubular configuration are radially expandable to intimately fit and secure to the blood vessel.

54. A method of repairing an abdominal aortic aneurysm (AAA), the method comprising steps of: (a) percutaneously introducing and advancing a guidewire into a first femoral artery, into a first iliac artery and then into a lumen of an aorta beyond area of the aneurysm; (b) assembling a neck attachment element in a collapsed state to be placed at about a distal end segment of a first deployment catheter, wherein the first deployment catheter having a guidewire lumen formed therein adapted to be fitted over the guidewire; (c) delivering the neck attachment element to a site of the aorta close to a renal artery ostium to provide an attachment seat of predetermined size approximating a diameter of the aorta lumen beyond and proximal to the area of the aneurysm; (d) deploying by means of self-expanding or balloon-expanding the neck attachment element to anchor securely the neck attachment element in place; (e) withdrawing the first deployment catheter; (f) assembling a first elongated tubular graft prosthesis in a collapsed state to be placed at about a distal end segment of a second deployment catheter, said first graft prosthesis having a proximal end, a distal end, and a continuous side wall extending between the proximal end to the distal end; (g) delivering the first graft prosthesis with the proximal end being positioned at about the neck attachment element and the distal end being positioned at about the first iliac artery; (h) deploying by means of anchoring the proximal end of the first graft prosthesis onto the neck attachment element while deploying the distal end in the first iliac artery; (i) percutaneously withdrawing the second deployment catheter.

55. The method according to claim 54, further comprising steps of: (a) assembling a second elongated tubular graft prosthesis in a collapsed state to be placed at about a distal end segment of a third deployment catheter, said second graft prosthesis having a proximal end, a distal end, and a continuous side wall extending between the proximal end to the distal end; (g) delivering the second graft prosthesis with the proximal end being positioned at about the neck attachment element and the distal end being positioned at about the second iliac artery; (h) deploying by means of anchoring the proximal end of the second graft prosthesis onto the neck attachment element while deploying the distal end in the second iliac artery; (i) percutaneously withdrawing the third deployment catheter.

56. The method according to claim 55, wherein the first proximal end of the first graft prosthesis is located at a substantial distance proximal to the second proximal end of the second graft prosthesis.

57. The method according to claim 55, wherein a circumferential area beyond two luminal openings of the proximal ends of the two graft prostheses is sealed so to allow blood flowing through said luminal openings of the graft prostheses.

58. The method according to claim 54, wherein the distal end of the first graft prosthesis is sized and configured, after deployment, to seal the graft lumen and iliac arteries from the aneurysm.

59. The method according to claim 54, with, the proximal end of the first graft prosthesis comprises barbs configured for securing to the neck attachment element.

60. The method according to claim 54, wherein the first graft prosthesis is reinforced with a metal mesh or stenting element at either end or both ends.

61. A radially expandable sheath as a guiding sheath, comprising a continuous integral sheath body with a thin wall that is radially expandable under outward forces, wherein the radially expandable sheath is characterized with substantially little or no axial stretchability or contraction from a first configuration of a compressed state to a second configuration of an expanded state.

62. A method of temporarily placing a hemostatic cuff at an incision of a blood vessel when inserting an endograft into a patient, the method comprising: (a) loading the hemostatic cuff on the expandable sheath of claim 1 at the first configuration; (b) inserting the compressed sheath through the incision into the blood vessel; (c) advancing the endograft into the blood vessel via a sheath lumen to expand the sheath to the second configuration; (d) holding the hemostatic cuff at proximity of the incision; and (e) removing the expanded sheath after the endograft and the cuff are properly positioned in place.

63. An endograft for treatment of an abdominal aortic aneurysm (AAA) comprising an impermeable section for excluding blood communication between a lumen of the endograft and a surrounding aneurysmal sac, and a porous section configured for placement across a renal artery ostium.

64. The endograft according to claim 63, wherein the endograft comprises a macro-porous sleeve that is longer than the impermeable section, the porous section being created by securing the macro-porous sleeve over at least a portion of the impermeable section.

65. A method for plugging an aneurysm in a patient, comprising introducing a PVA sponge substrate with a porosity at a first configuration of a dried compressed state into said aneurysm and allowing said sponge substrate to expand in situ to a second configuration to fill the aneurysm.

66. The method according to claim 65, wherein the sponge substrate is incorporated with a radiopaque marker for external visualization.

67. The method according to claim 65, wherein the aneurysm is an aneurysmal blood vessel, the sponge substrate comprising a conformable pair of spongy endo-plugs for treatment of the aneurysmal vessel, wherein the endo-plugs are compressed in a first configuration for delivery to the vessel and expand via re-hydration to a second configuration to plug the vessel.

68. The method according to claim 67, wherein each endo-plug has a through lumen.

69. The method according to claim 67, wherein each endo-plug has a matching flat surface facing each other.

70. The method according to claim 67, wherein each endo-plug has a matching ribbed surface to provide interlocked seal in the blood vessel after re-hydration.

71. The method according to claim 65, wherein the aneurysm is an aneurysmal blood vessel, the sponge substrate comprising a spongy endo-plug, wherein the endo-plug is compressed in a first configuration for delivery to the vessel and expands to a second configuration via an embedded shape memory Nitinol wire.

72. The method according to claim 65, wherein the sponge substrate comprises a spongy endo-plug with an anchoring means for securing the endo-plug in place without undue migration.

73. A method of treatment of an abdominal aortic aneurysm (AAA), comprising inserting soft, thrombogenic pipe-cleaner like soft filler material into a AAA sac with a delivery catheter.

74. The method according to claim 73, wherein the material is made of PVA (polyvinyl alcohol) or Dacron (polyester) thread that enhances thrombogenic properties.

75. A balloon endograft comprising: a neck attachment member, a body and at least one distal end, wherein the endograft comprises double layers and a space between the layers, the space being configured to be filled with inflatable fluid or hardenable foam to inflate the balloon endograft.

76. The balloon endograft according to claim 75, wherein the endograft is characterized with no stiff component prior to inflating said balloon endograft.

77. The balloon endograft according to claim 75, wherein the body comprises two inflatable tubes, each inflatable tube having a proximal end secured to the neck attachment member, a distal end, and double layers with a space between the layers.

78. The balloon endograft according to claim 75, wherein the body is configured in a corrugated configuration.

79. The balloon endograft according to claim 75, wherein the neck attachment member comprises two inflatable neck attachment rings and at least two connecting units that connect the two rings, wherein said neck attachment rings are inflatable to anchor securely at wall of a blood vessel.
